(19) United States
(12) Patent Application Publication
(10) Pub. No.: US 2025/0177278 A1
(43) Pub. Date: Jun. 5, 2025

(54) TREATMENT OF UPPER FACIAL LINES

(71) Applicant: IPSEN BIOPHARM LIMITED, Wrexham (GB)

(72) Inventors: Nicolae Grigore, Wrexham (GB); Laurent Pons, Wrexham (GB); Katie Ruffle, Wrexham (GB); Philippe Picaut, Wrexham (GB)

(21) Appl. No.: 18/851,525

(22) PCT Filed: Apr. 28, 2023
(86) PCT No.: PCT/GB2023/051152

§ 371 Date: Sep. 26, 2024

(30) Foreign Application Priority Data
Apr. 29, 2022 (GB) 2206362.2

(51) Int. Cl. A61K 8/66; A61Q 19/08

(52) U.S. Cl. CPC A61K 8/66; A61Q 19/08; A61K 2800/91

(57) ABSTRACT

The present invention provides a modified BoNT/A for use in treating facial lines, wherein the modified BoNT/A is administered by intramuscular injection at a plurality of sites of the face of the subject, wherein the modified BoNT/A is administered by way of a unit dose of greater than 1754 pg (e.g. greater than 1800 pg) of modified BoNT/A per site, wherein the plurality of sites are selected from: up to two sites of a corrugator muscle and one site of a procerus muscle for treating glabellar lines, up to five sites of a frontalis muscle for treating forehead lines, and up to three sites of an orbicularis oculi muscle for treating lateral canthal lines, wherein the total dose of modified BoNT/A administered during the treatment is up to 88,000 pg, and wherein the modified BoNT/A comprises a BoNT/A light-chain and translocation domain (H_{N} domain), and a BoNT/B receptor binding domain (H_{C} domain).

### FIELD OF THE INVENTION

The present invention relates to cosmetic treatment (e.g. for treating hyperfunctional facial lines). In more detail, the invention provides methods of cosmetic treatment comprising administration of longer acting neurotoxins and more particularly, to a method of treating cosmetic irregularities using longer acting botulinum neurotoxins.

### BACKGROUND

Hyperfunctional facial lines are common aesthetic irregularities that can include but are not limited to; glabellar lines, lateral canthal lines, forehead lines and wrinkles, crow's feet, eyebrow lines, nasolabial folds, lip lines and marionette lines. Upper facial lines can occur in the forehead, glabellar and the lateral orbital areas. Wrinkling may also appear in the glabellar and forehead areas due to the expression of frowning, whilst wrinkling may appear in the lateral canthal areas due to the expression of smiling. Excessively prominent lines in this area are often misinterpreted as fatigue causing subjects considerable distress regarding their appearance. Instead, excessively prominent facial lines appear as a result from the functional pull of the underlying muscles, which eventually creases the skin. In the glabellar complex, these muscles include the corrugator supercilli, the procerus and the depressor supercilli, whilst the orbicularis oculi muscle is responsible for the production of lateral canthal lines.

Over the last few decades, as the aging population continues to grow, there has been an increasing demand for cosmetic procedures to reverse the appearance of advancing age, particularly in the facial area. This increasing preoccupation with physical appearance has led to the development of many different products and procedures, such as surgery, resurfacing of various types and the use of filling agents. Because none of these methods are entirely risk free, continuous research is necessary to provide the safest and most effective methods for treatment of the aging face.

Although there are five factors that interplay in the production of what is known as the aging face, there are primarily two factors (the skin and the underlying muscles) interacting to produce the more significant lines and folds, as opposed to wrinkles. Many therapies have evolved to treat wrinkles and the skin factors of the lines and folds, among them, various types of resurfacing, dermatologic products and injections for soft tissue augmentation.

An alternative cosmetic procedure for treating the appearance of facial lines is the administration of neurotoxin, specifically botulinum neurotoxin, to the underlying muscles of facial skin. Dysport® is a medicinal product containing drug substance BoNT/A haemagluttinin complex (BTX-A-HAC) isolated and purified from Clostridium botulinum type A strain. Several other medicinal BoNT/A products naturally produced by Clostridium botulinum are also on the market.

BoNT/A selectively inhibits the release of acetylcholine from the presynaptic nerve terminals and thus blocks cholinergic transmission at the neuromuscular junction inducing a reduction in the muscle contraction and muscle tone, causing the injected muscles to relax.

Dysport® is approved for the treatment of glabellar lines with a maximum total dose of up to 50 Units to be administered across the corrugator muscles and procerus muscle (see FIG. 1). The clinical effect of Dysport may last up to four months. Repeat dosing in clinical studies demonstrated continued efficacy with up to four repeated administrations. However, Dysport should be administered no more frequently than every three months. Dysport has so far not been FDA approved for the treatment of forehead lines and lateral canthal lines.

To avoid systemic neurological effects, many clostridial toxin based cosmetic treatments utilise direct administration of the clostridial toxin therapeutic to a given target site (such as a target tissue). A problem when administering clostridial toxin-based therapeutics in this fashion is the spread of toxin away from the administration site and into surrounding tissue or systemic circulation. The diffusion of toxin away from the target tissue is believed to be responsible for undesirable side effects that in extreme cases may be life threatening. This can be a particular concern when using clostridial toxin therapeutics (such as BoNT therapeutics) at high doses, concentrations and injection volumes. Adverse effects associated with this problem that have been reported for commercial BoNT/A therapeutics include asthenia, generalised muscle weakness, diplopia, ptosis, dysphagia, dysphonia, dysarthria, urinary incontinence, and breathing difficulties. Swallowing and breathing difficulties can be life threatening and there have been reported deaths related to the spread of toxin effects.

The present invention overcomes one or more of the above-mentioned problems.

### SUMMARY OF THE INVENTION

The present inventors have surprisingly found that a modified BoNT/A finds particular utility in cosmetic treatment of facial lines. A modified BoNT/A may comprise a BoNT/A light-chain and translocation domain and a BoNT/B receptor binding domain (H_{C} domain), which results in a modified BoNT/A that exhibits increased retention at (reduced diffusion away from) a site of administration and increased duration of action (e.g. 6-9 months). Advantageously, modified BoNT/A has a safety profile that is improved when compared to unmodified BoNT/A (e.g. Dysport®). This improved safety profile may be expressed by the high Safety Ratio described herein for the modified BoNT/A. The modified BoNT/A may comprise one or more modifications of surface exposed amino acid residues resulting in an increased net positive charge. The increased charge promotes electrostatic interactions between the polypeptide and anionic extracellular components, thereby promoting binding between the polypeptide and cell surface. In turn this increases retention at (reduces diffusion away from) a site of administration and results in an increased duration of action (e.g. 6-9 months).

Based on the pre-clinical data herein (see Example 11) it has been shown that a higher total amount of modified BoNT/A can be administered to a subject while achieving a similar safety profile to unmodified BoNT/A (e.g. Dysport®) while at such high doses. Thus, modified BoNT/A can be injected at a greater number of muscles/sites in the cosmetic treatment of facial lines before reaching the maximum total dose. This is a significant and advantageous finding and yields an improved cosmetic treatment of facial lines while providing clinicians with a greater range of treatment options. The treatment may be improved in that it provides for longer-lasting treatment (resulting in less frequent administration) when compared to treatment with unmodified BoNT/A (e.g. Dysport®) and/or is capable of being tailored for the subject, for example, enabling the clinician to administer at particular sites according to the subject's aesthetic requirements. The treatment of the invention is improved compared to conventional treatment regimens.

Moreover, the present invention provides a convenient, safe, and effective single dose unit as well as a total (maximum) dosage that can safely be administered in a single treatment. The present invention also provides a corresponding guide to the number of times at which said dose unit can be administered to a muscle (including the number of injection sites per muscle) without resultant patient toxicity. Treatment in accordance with the present invention is thus much less complicated for the clinician and helps avoid under-dosing and/or over-dosing.

### DETAILED DESCRIPTION

In one aspect the invention provides a modified BoNT/A for use in treating facial lines (e.g. for a longer duration than that treated by an unmodified BoNT/A (e.g. SEQ ID NO: 2)), wherein the modified BoNT/A is administered by intramuscular injection at a plurality of sites of the face of the subject,
wherein the modified BoNT/A is administered by way of a unit dose of greater than 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites an orbicularis oculi muscle for treating lateral canthal lines (e.g. up to three sites at the external part of an orbicularis oculi muscle for treating lateral canthal lines),
wherein the total dose of modified BoNT/A administered during the treatment is up to 3,661 Units, and
wherein the modified BoNT/A comprises a BoNT/A light-chain and translocation domain, and a BoNT/B receptor binding domain (H_{C} domain).

Corresponding uses (in the manufacture of a medicament) and methods of treating facial liens are also provided.

In a related aspect, the invention provides a method for treating facial lines (e.g. for a longer duration than that treated by an unmodified BoNT/A (e.g. SEQ ID NO: 2)), the method comprising administering a modified botulinum neurotoxin A (BoNT/A) by intramuscular injection at a plurality of sites of the face of the subject,
wherein the modified BoNT/A is administered by way of a unit dose of greater than 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
wherein the total dose of modified BoNT/A administered during the treatment is up to 3,661 Units, and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating facial lines (e.g. for a longer duration than that treated by an unmodified BoNT/A (e.g. SEQ ID NO: 2)), wherein the modified BoNT/A is administered by intramuscular injection at a plurality of sites of the face of the subject,
wherein the modified BoNT/A is administered by way of a unit dose of greater than 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
wherein the total dose of modified BoNT/A administered during the treatment is up to 3,661 Units, and
wherein the modified BoNT/A comprises a BoNT/A light-chain and translocation domain, and a BoNT/B receptor binding domain (H_{C} domain).

In one aspect, the invention provides a modified BoNT/A for use in treating facial lines (e.g. for a longer duration than that treated by an unmodified BoNT/A (e.g. SEQ ID NO: 2)), wherein the modified BoNT/A is administered by intramuscular injection at a plurality of sites of the face of the subject,
wherein the modified BoNT/A is administered by way of a unit dose of greater than 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
   preferably wherein the plurality of sites comprise (or consist of) up to two sites of a corrugator muscle and one site of a procerus muscle for treating glabellar lines,
wherein the total dose of modified BoNT/A administered during the treatment is up to 1144 Units (e.g. up to 1019 Units), and
wherein the modified BoNT/A comprises a BoNT/A light-chain and translocation domain, and a BoNT/B receptor binding domain (H_{C} domain).

In a related aspect, the invention provides a method for treating facial lines (e.g. for a longer duration than that treated by an unmodified BoNT/A (e.g. SEQ ID NO: 2)), the method comprising administering a modified botulinum neurotoxin A (BoNT/A) by intramuscular injection at a plurality of sites of the face of the subject,
wherein the modified BoNT/A is administered by way of a unit dose of greater than 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
   preferably wherein the plurality of sites comprise (or consist of) up to two sites of a corrugator muscle and one site of a procerus muscle for treating glabellar lines,
wherein the total dose of modified BoNT/A administered during the treatment is up to up to 1144 Units (e.g. up to 1019 Units), and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating facial lines (e.g. for a longer duration than that treated by an unmodified BoNT/A (e.g. SEQ ID NO: 2)), wherein the modified BoNT/A is administered by intramuscular injection at a plurality of sites of the face of the subject,
wherein the modified BoNT/A is administered by way of a unit dose of greater than 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
   preferably wherein the plurality of sites comprise (or consist of) up to two sites of a corrugator muscle and one site of a procerus muscle for treating glabellar lines,
wherein the total dose of modified BoNT/A administered during the treatment is up to 1144 Units (e.g. up to 1019 Units), and
wherein the modified BoNT/A comprises a BoNT/A light-chain and translocation domain, and a BoNT/B receptor binding domain (H_{C} domain).

In another aspect, the invention provides a modified BoNT/A for use in treating facial lines (e.g. for a longer duration than that treated by an unmodified BoNT/A (e.g. SEQ ID NO: 2)), wherein the modified BoNT/A is administered by intramuscular injection at a plurality of sites of the face of the subject,
wherein the modified BoNT/A is administered by way of a unit dose of greater than 1754 pg (e.g. greater than 1800 pg) of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
wherein the total dose of modified BoNT/A administered during the treatment is up to 88,000 (e.g. up to 80,000 pg), and
wherein the modified BoNT/A comprises a BoNT/A light-chain and translocation domain, and a BoNT/B receptor binding domain (H_{C} domain).

In a related aspect, the invention provides a method for treating facial lines (e.g. for a longer duration than that treated by an unmodified BoNT/A (e.g. SEQ ID NO: 2)), the method comprising administering a modified botulinum neurotoxin A (BoNT/A) by intramuscular injection at a plurality of sites of the face of the subject,
wherein the modified BoNT/A is administered by way of a unit dose of greater than 1754 pg (e.g. greater than 1800 pg) of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
wherein the total dose of modified BoNT/A administered during the treatment is up to 88,000 (e.g. up to 80,000 pg), and
wherein the modified BoNT/A comprises a BoNT/A light-chain and translocation domain, and a BoNT/B receptor binding domain (H_{C} domain).

In another related aspect, the invention provides use of a modified botulinum neurotoxin A (BoNT/A) in the manufacture of a medicament for treating facial lines (e.g. for a longer duration than that treated by an unmodified BoNT/A (e.g. SEQ ID NO: 2)), wherein the modified BoNT/A is administered by intramuscular injection at a plurality of sites of the face of the subject,
wherein the modified BoNT/A is administered by way of a unit dose of greater than 1754 pg (preferably greater than 1800 pg, more preferably greater than 2555 pg) of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
wherein the total dose of modified BoNT/A administered during the treatment is up to 88,000 (e.g. up to 80,000 pg), and
wherein the modified BoNT/A comprises a BoNT/A light-chain and translocation domain, and a BoNT/B receptor binding domain (H_{C} domain).

In another aspect, the invention provides a modified BoNT/A for use in treating facial lines (e.g. for a longer duration than that treated by an unmodified BoNT/A (e.g. SEQ ID NO: 2)), wherein the modified BoNT/A is administered by intramuscular injection at a plurality of sites of the face of the subject,
wherein the modified BoNT/A is administered by way of a unit dose of greater than 1754 pg (e.g. greater than 1800 pg) of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
   preferably wherein the plurality of sites comprise (or consist of) up to two sites of a corrugator muscle and one site of a procerus muscle for treating glabellar lines,
wherein the total dose of modified BoNT/A administered during the treatment is up to 27,500 (e.g. up to 25,000 pg), and
wherein the modified BoNT/A comprises a BoNT/A light-chain and translocation domain, and a BoNT/B receptor binding domain (H_{C} domain).

In a related aspect, the invention provides a method for treating facial lines (e.g. for a longer duration than that treated by an unmodified BoNT/A (e.g. SEQ ID NO: 2)), the method comprising administering a modified botulinum neurotoxin A (BoNT/A) by intramuscular injection at a plurality of sites of the face of the subject,
wherein the modified BoNT/A is administered by way of a unit dose of greater than 1754 pg (e.g. greater than 1800 pg) of modified BoNT/A per site,
wherein the plurality of sites are selected from:
up to two sites of a corrugator muscle and one site of a procerus muscle for treating glabellar lines,
up to five sites of a frontalis muscle for treating forehead lines; and
up to three sites of an orbicularis oculi muscle for treating lateral canthal lines (e.g. up to three sites at the external part of an orbicularis oculi muscle for treating lateral canthal lines),
preferably wherein the plurality of sites comprise (of consist of) up to two sites of a corrugator muscle and one site of a procerus muscle for treating glabellar lines,
wherein the total dose of modified BoNT/A administered during the treatment is up to 27,500 (e.g. up to 25,000 pg), and
wherein the modified BoNT/A comprises a BoNT/A light-chain and translocation domain, and a BoNT/B receptor binding domain (HC domain).

In another related aspect, the invention provides use of a modified botulinum neurotoxin A (BoNT/A) in the manufacture of a medicament for treating facial lines (e.g. for a longer duration than that treated by an unmodified BoNT/A (e.g. SEQ ID NO: 2)), wherein the modified BoNT/A is administered by intramuscular injection at a plurality of sites of the face of the subject,
wherein the modified BoNT/A is administered by way of a unit dose of greater than 1754 pg (preferably greater than 1800 pg, more preferably greater than 2555 pg) of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites of an orbicularis oculi muscle for treating lateral canthal lines (e.g. up to three sites at the external part of an orbicularis oculi muscle for treating lateral canthal lines),
   preferably the plurality of sites comprise (or consist of) up to two sites of a corrugator muscle and one site of a procerus muscle for treating glabellar lines,
wherein the total dose of modified BoNT/A administered during the treatment is up to 27,500 pg (e.g. up to 25,000 pg), and
wherein the modified BoNT/A comprises a BoNT/A light-chain and translocation domain, and a BoNT/B receptor binding domain (H_{C} domain).

Said facial lines may most preferably be referred to as “upper facial lines” and (as reflected by the aspects above) include glabellar lines, forehead lines and lateral canthal lines. The invention may involve treating any one of said facial lines (e.g. upper facial lines), or any combination therefore. For example, all three of glabellar lines, forehead lines and lateral canthal lines may be treated by methods of the invention. That being said, a particular type of facial line may be targeted. For example, the facial lines treated may be glabellar lines. Additionally, or alternatively, the facial lines treated may be forehead lines. Additionally, or alternatively, the facial lines treated may be lateral canthal lines.

Particular combinations of facial lines may be treated by the invention. Examples of facial line combinations include:
glabellar lines and forehead lines;
glabellar lines and lateral canthal lines;
forehead lines and lateral canthal lines.

A preferred combination of facial lines treated by the invention may be glabellar lines and lateral canthal lines.

Reference to “muscle” means an affected muscle that contributes (e.g. via excess tightness, tension, or tone) to an underlying aesthetic condition that the present invention addresses.

A first group of muscles is defined by a procerus muscle, a second group of muscles is defined by a corrugator muscle, a third group of muscles is defined by an orbicularis oculi muscle, and a fourth group of muscles is defined by a frontalis muscle.

Throughout this disclosure, the term “up to two sites of a corrugator muscle” may be used interchangeably with the term “up to two sites per corrugator muscle”, noting that there are two corrugator muscles. As such, the modified BoNT/A may be administered at up to four sites of the corrugator muscles, e.g. with the proviso that the modified BoNT/A is administered to up to two sites per corrugator muscle.

The corrugator is also known as the corrugator supercilli.

The sites of the orbicularis occuli (to which a unit dose of modified BoNT/A is administered) are preferably at the external part of an orbicularis oculi muscle. The term “the external part of an orbicularis oculi muscle” may be used interchangeably with the term “the lateral upper orbicularis oculi muscle”.

Administering at “up to three sites of an orbicularis oculi muscle” may comprise:
a) administering a unit dose of the modified BoNT/A to the lateral upper orbicularis oculi muscle (e.g. proximal to a first eye of the subject);
b) administering a unit dose of the modified BoNT/A to the medial upper orbicularis oculi muscle (e.g. proximal to the first eye of the subject); and
c) administering a unit dose of the modified BoNT/A to the lateral lower orbicularis oculi muscle (e.g. proximal to the first eye of the subject).

Administering at “up to three sites of an orbicularis oculi muscle” may comprise:
a) administering a unit dose of the modified BoNT/A to the lateral upper orbicularis oculi muscle (e.g. proximal to a first eye of the subject);
b) administering a unit dose of the modified BoNT/A to a first site of the lateral lower orbicularis oculi muscle (e.g. proximal to the first eye of the subject); and
c) administering a unit dose of the modified BoNT/A to a second (different) site of the lateral lower orbicularis oculi muscle (e.g. proximal to the first eye of the subject).

Throughout this disclosure, the term “up to three sites an orbicularis oculi muscle” may be used interchangeably with the term “up to three sites per orbicularis oculi muscle”, noting that there are two orbicularis oculi muscles. Similarly, throughout this disclosure, the term “up to three sites at the external part of an orbicularis oculi muscle” may be used interchangeably with the term “up to three sites per orbicularis oculi muscle, at the external part of the orbicularis oculi muscle”, noting that there are two orbicularis oculi muscles. As such, the modified BoNT/A may be administered at up to six sites of the orbicularis oculi muscles, e.g. with the proviso that the modified BoNT/A is administered to up to three sites per orbicularis oculi muscle.

Administering at “up to three sites of an orbicularis oculi muscle” may comprise:
a) administering a unit dose of the modified BoNT/A per lateral upper orbicularis oculi muscle;
b) administering a unit dose of the modified BoNT/A per medial upper orbicularis oculi muscle; and
c) administering a unit dose of the modified BoNT/A per lateral lower orbicularis oculi muscle.

Administering at “up to three sites of an orbicularis oculi muscle” may comprise:
a) administering a unit dose of the modified BoNT/A per lateral upper orbicularis oculi muscle (e.g. proximal to a first eye of the subject);
b) administering a unit dose of the modified BoNT/A to a first site per lateral lower orbicularis oculi muscle (e.g. proximal to the first eye of the subject); and
c) administering a unit dose of the modified BoNT/A to a second (different) site per lateral lower orbicularis oculi muscle (e.g. proximal to the first eye of the subject).

Reference to an “upper” orbicularis oculi muscle refers to an orbicularis oculi muscle of an upper eyelid. Similarly, reference to a “lower” orbicularis oculi muscle refers an orbicularis oculi muscle of a lower eyelid.

The skilled person understands that the term “medial” (e.g. in the context of anatomy) means toward the midline of the body. Similarly, the skilled person understands that the term “lateral” (e.g. in the context of anatomy) means away from the midline of the body. Thus:
the “lateral” upper orbicularis oculi muscle refers to a site of the orbicularis oculi muscle, of an upper eyelid, that is positioned away from the midline of the body
the “medial” upper orbicularis oculi muscle refers to a site of the orbicularis oculi muscle, of an upper eyelid, that is positioned toward the midline of the body
the “lateral” lower orbicularis oculi muscle refers to a site of the orbicularis oculi, of a lower eyelid, that is positioned away from the midline of the body.

The term “lateral upper orbicularis oculi muscle” may be used synonymously with the term “the external part of an orbicularis oculi muscle of an upper eyelid”. The term “medial upper orbicularis oculi muscle” may be used synonymously with the term “the inner part of an orbicularis oculi muscle of an upper eyelid”. The term “lateral lower orbicularis oculi muscle” may be used synonymously with the term “the external part of an orbicularis oculi muscle of a lower eyelid”.

An orbicularis oculi muscle comprises a “pretarsal portion” and a “preseptal portion” (either of which can be injected into). An orbicularis oculi muscle comprise an “orbital portion”.

Preferably, a unit dose that is administered to the orbicularis oculi muscle may be administered to an orbital portion of said muscle, more preferably to an orbital portion at an external part (e.g. lateral part) of the orbicularis oculi muscle. Thus, the plurality of sites may comprise (or consist of) up to three sites of an orbital portion at an external part (e.g. lateral part) of an orbicularis oculi muscle for treating lateral canthal lines.

Administration to a lateral upper orbicularis oculi muscle may mean administering to a lateral “pretarsal” upper orbicularis oculi muscle, or to a lateral “preseptal” upper orbicularis oculi muscle.

Administration to a medial upper orbicularis oculi muscle may mean administration to a medial upper “pretarsal” orbicularis oculi muscle, or to a medial upper “preseptal” orbicularis oculi muscle.

Administration to a lateral lower orbicularis oculi muscle may mean administration to a lateral lower “pretarsal” orbicularis oculi muscle, or to a lateral lower “preseptal” orbicularis oculi muscle.

When administering to a lateral upper orbicularis oculi muscle, said unit dose may be administered to the preseptal portion (in other words, to a lateral “preseptal” upper orbicularis oculi muscle). When administering to a medial upper orbicularis oculi muscle, said unit dose may be administered to the preseptal portion (in other words, to a medial upper “preseptal” orbicularis oculi muscle). When administering to a lateral lower orbicularis oculi muscle, said unit dose may be administered to the preseptal portion (in other words, to a lateral lower “preseptal” orbicularis oculi muscle).

The number of sites to which a modified BoNT/A is administered may vary depending on the facial line to be treated.

For example, the facial lines to be treated may be glabellar lines, and the term “plurality of sites” may mean up to three sites, wherein the plurality of sites comprise (or consist of) up to two sites of a corrugator muscle and one site of a procerus muscle for treating glabellar lines. It is preferred that two unit doses of modified BoNT/A are administered at to two different sites of a corrugator and one unit dose is administered to one site of a procerus for treating glabellar lines (e.g. such that three unit doses are administered at three different sites).

Glabellar lines on one side of the face may be treated, and the “plurality of sites” may mean up to three sites as defined above, such that the subject receives up to three unit doses of a modified BoNT/A.

The facial lines to be treated may be glabellar lines, and the term “plurality of sites” may mean up to six sites, wherein a unit dose of modified BoNT/A is at up to two sites per corrugator muscle and one site of a procerus muscle for treating glabellar lines. It is preferred that two unit doses of modified BoNT/A are administered to two different sites per corrugator and one unit dose is administered to one site of the procerus for treating glabellar lines (e.g. such that five unit doses are administered at five different sites). Glabellar lines on both sides of the face may be treated, and the “plurality of sites” may mean up to six sites as defined above, such that the subject receives up to six unit doses of a modified BoNT/A.

The facial lines to be treated may be forehead lines, and the term “plurality of sites” may mean up to two, three, four or five sites of a frontalis muscle for treating forehead lines. It is preferred that that five unit doses are administered to five different sites of the frontalis for treating forehead lines (e.g. such that five unit doses are administered at five different sites).

The facial lines to be treated may be lateral canthal lines, and the term “plurality of sites” may mean up to two or three sites of an orbicularis oculi muscle (e.g. up to two or three sites at an external part of an orbicularis oculi muscle). It is preferred that three unit doses of modified BoNT/A are administered to three different sites of an orbicularis oculi muscle for treating lateral canthal lines (e.g. such that three unit doses are administered at three different sites). Lateral canthal lines on one side of the face may be treated, and the “plurality of sites” may mean up to three sites as defined above, such that the subject receives up to three unit doses of a modified BoNT/A.

The facial lines to be treated may be lateral canthal lines, and the term “plurality of sites” may mean up to two or three sites per orbicularis oculi muscle (e.g. up to two or three sites per orbicularis oculi muscle, at an external part of the orbicularis oculi muscle). It is preferred that three unit doses of modified BoNT/A are administered to three different sites per orbicularis oculi muscle for treating lateral canthal lines (e.g. such that six unit doses are administered at six different sites). Lateral canthal lines on both sides of the face may be treated, and the “plurality of sites” may mean up to six sites as defined above, such that the subject receives up to six unit doses of a modified BoNT/A.

The facial lines to be treated may be glabellar lines and forehead lines, and the term “plurality of sites” may mean up to eight sites, wherein the plurality of sites comprise (or consist of) up to two sites of a corrugator muscle and one site of a procerus muscle for treating glabellar lines, and up to five sites of a frontalis muscle for treating forehead lines. It is preferred that two unit doses of modified BoNT/A are administered at two different sites of a corrugator and one unit dose is administered to one site of a procerus for treating glabellar lines, and five unit doses are administered to five different sites of a frontalis muscles for treating forehead lines (e.g. such that eight unit doses are administered at eight different sites).

The facial lines to be treated may be glabellar lines and forehead lines, and the term “plurality of sites” may mean up to ten sites, wherein the plurality of sites comprise (or consist of) up to two sites per corrugator muscle and one site of a procerus muscle for treating glabellar lines, and up to five sites of a frontalis muscle for treating forehead lines. It is preferred that two unit doses of modified BoNT/A are administered at two different sites per corrugator and one unit dose is administered to one site of a procerus for treating glabellar lines, and five unit doses are administered to five different sites of a frontalis muscles for treating forehead lines (e.g. such that ten unit doses are administered at ten different sites).

The facial lines to be treated may be glabellar lines and lateral canthal lines, and the term “plurality of sites” may mean up to three, four, five or six sites, wherein the plurality of sites comprise (or consist of) up to two sites of a corrugator muscle and one site of a procerus muscle for treating glabellar lines, and up to two or three sites of an orbicularis oculi muscle (e.g. up to two or three sites at an external part of an orbicularis oculi muscle) for treating lateral canthal lines. It is preferred that two unit doses of modified BoNT/A are administered to two different sites of a corrugator and one unit dose is administered to one site of a procerus for treating glabellar lines, and three unit doses of modified BoNT/A are administered to three different sites of an orbicularis oculi muscle for treating lateral canthal lines (e.g. such that six unit doses are administered at six different sites).

The facial lines to be treated may be glabellar lines and lateral canthal lines, and the term “plurality of sites” may mean up to three, four, five, six, seven, eight, nine, ten or eleven sites, wherein the plurality of sites comprise (or consist of) up to two sites per corrugator muscle and one site of a procerus muscle for treating glabellar lines, and up to two or three sites per orbicularis oculi muscle (e.g. up to two or three sites per orbicularis oculi muscle, at an external part of an orbicularis oculi muscle) for treating lateral canthal lines. It is preferred that two unit doses of modified BoNT/A are administered to two different sites per corrugator and one unit dose is administered to one site of a procerus for treating glabellar lines, and three unit doses of modified BoNT/A are administered to three different sites per orbicularis oculi muscle for treating lateral canthal lines (e.g. such that eleven unit doses are administered at eleven different sites).

The facial lines to be treated may be forehead lines and lateral canthal lines, and the term “plurality of sites” may mean up to four, five, six, seven or eight sites, wherein the plurality of sites comprise (or consist of) up to two, three, four or five sites of a frontalis muscle for treating forehead lines, and up to two or three sites of an orbicularis oculi muscle (e.g. up to two or three sites at an external part of an orbicularis oculi muscle) for treating lateral canthal lines. It is preferred that five unit doses are administered to five different sites of a frontalis muscles for treating forehead lines, and three unit doses of modified BoNT/A are administered to three different sites of an orbicularis oculi muscle for treating lateral canthal lines (e.g. such that eight unit doses are administered at eight different sites).

The facial lines to be treated may be forehead lines and lateral canthal lines, and the term “plurality of sites” may mean up to four, five, six, seven, eight, nine, ten or eleven sites, wherein the plurality of sites comprise (or consist of) up to two, three, four or five sites of a frontalis muscle for treating forehead lines, and up to two or three sites per orbicularis oculi muscle (e.g. up to two or three sites at an external part per orbicularis oculi muscle) for treating lateral canthal lines. It is preferred that five unit doses are administered to five different sites of a frontalis muscles for treating forehead lines, and three unit doses of modified BoNT/A are administered to three different sites per orbicularis oculi muscle for treating lateral canthal lines (e.g. such that eleven unit doses are administered at eleven different sites).

The facial lines to be treated may be glabellar lines, forehead lines and lateral canthal lines, and the term “plurality of sites” may mean up to four, five, six, seven, eight, nine, ten, eleven, twelve, thirteen, fourteen, fifteen or sixteen sites, wherein the plurality of sites comprise (or consist of)
up to two sites of a corrugator muscle and one site of a procerus muscle for treating glabellar lines,
up to two, three, four or five sites of a frontalis muscle for treating forehead lines, and
up to two or three sites of an orbicularis oculi muscle (e.g. up to two or three sites at an external part of an orbicularis oculi muscle) for treating lateral canthal lines.

It is preferred that:
two unit doses of modified BoNT/A are administered to two different sites of a corrugator and one unit dose is administered to one site of a procerus for treating glabellar lines,
five unit doses are administered to five different sites of a frontalis muscles for treating forehead lines, and
three unit doses of modified BoNT/A are administered to three different sites of an orbicularis oculi muscle (e.g. an external part thereof) for treating lateral canthal lines
(e.g. such that eleven unit doses are administered at eleven different sites).

The facial lines to be treated may be glabellar lines, forehead lines and lateral canthal lines, and the term “plurality of sites” may mean up to four, five, six, seven, eight, nine, ten, eleven, twelve, thirteen, fourteen, fifteen or sixteen sites, wherein the plurality of sites comprise (or consist of):
up to two sites per corrugator muscle and one site of a procerus muscle for treating glabellar lines,
up to two, three, four or five sites of a frontalis muscle for treating forehead lines, and
up to two or three sites per orbicularis oculi muscle (e.g. up to two or three sites at an external part per orbicularis oculi muscle) for treating lateral canthal lines.

It is preferred that:
two unit doses of modified BoNT/A are administered to two different sites per corrugator and one unit dose is administered to one site of a procerus for treating glabellar lines,
five unit doses are administered to five different sites of a frontalis muscles for treating forehead lines, and
three unit doses of modified BoNT/A are administered to three different sites per orbicularis oculi muscle (e.g. an external part thereof) for treating lateral canthal lines
(e.g. such that sixteen unit doses are administered at eleven different sites).

Thus, the term “a plurality of sites” may mean up to two, three, four, five, six, seven, eight, nine, ten, eleven, twelve, thirteen, fourteen, fifteen or sixteen sites. Preferably, the term “a plurality of sites” may refer to “up to 16 sites”.

The term “a plurality of sites” may mean two, three, four, five, six, seven, eight, nine, ten, eleven, twelve, thirteen, fourteen, fifteen or sixteen sites. Preferably, the term “a plurality of sites” may refer to 16 sites.

Thus, the modified BoNT/A may be administered by intramuscular injection at up to 16 sites of the face of the subject, for example
up to four sites of the corrugator muscles (e.g. with the proviso that the modified BoNT/A is administered to up to two sites per corrugator muscle) and one site of a procerus muscle for treating glabellar line;
up to five sites of a frontalis muscle for treating forehead lines; and
up to six sites of the orbicularis oculi muscles (e.g. with the proviso that the modified BoNT/A is administered to up to three sites per orbicularis oculi muscle) for treating lateral canthal lines.

The plurality of sites may be on the same muscle group (eg. right and left corrugator muscle, right and left orbicularis oculi muscle). Similarly, the plurality of sites may be on the same muscle (eg. right corrugator muscle or left corrugator muscle; or right orbicularis oculi muscle or left orbicularis oculi muscle). Similarly, the plurality of sites may be on a combination of said muscle and muscle groups. Thus, a personalised treatment regimen may be provided.

Potency of a modified BoNT/A for use according to the invention is preferably determined by a mouse LD₅₀ assay according to standard techniques. In said assay, 1 Unit is defined as an amount of the modified BoNT/A that corresponds to the calculated median lethal dose (LD₅₀) in mice. Preferably, the calculated median lethal intraperitoneal dose in mice.

In particular, an amount of a modified BoNT/A that corresponds to 1 Unit in said assay is preferably 24.04 pg.

The term “up to” when used in reference to a value (e.g. up to 3661 Units) means up to and including the value recited. Thus, as an example, reference to administering “up to 3661 Units” of modified BoNT/A encompasses administration of 3661 Units of modified BoNT/A as well as administration of less than 3661 Units of modified BoNT/A. As another example, reference to administering “up to 80,000 pg” of modified BoNT/A encompasses administration of 80,000 pg of modified BoNT/A as well as administration of less than 80,000 pg of modified BoNT/A.

A dose of modified BoNT/A is administered by intramuscular injection at a plurality of sites. A single unit dose is administered per site. The term “a (single) unit dose is administered” means substantially all of a (single) unit dose is administered. Preferably at most only a residual amount (e.g. up to 1%, 0.1% or 0.01%) of the (single) unit dose may remain in a vial in which the modified BoNT/A has been reconstituted. However, preferably all of a (single) unit dose is administered. Depending on the muscle group, a (single) unit dose is typically administered to a muscle selected from the first group (described herein), a 2× unit is typically administered to a muscle selected from the second group (described herein), a 3× unit is typically administered to a muscle selected from the third group (described herein), and a 5× unit is typically administered to a muscle selected from the fourth group (described herein).

The unit dose can be expressed in terms of Units of modified BoNT/A. For example, 24.04 pg may correspond to 1 Unit of modified BoNT/A. By way of example, 2404 pg of BoNT/A may be referred to as 100 Units of modified BoNT/A.

The unit dose may be greater than 73 Units of modified BoNT/A. An upper limit of the unit dose range may be 3,661, 3000, 2500, 2000, 1500, 1000, 500 or 100 Units of modified BoNT/A. A lower limit of the unit dose range may be 75, 100, 150, 200, 300 or 500 Units of modified BoNT/A. Preferably, the unit dose of modified BoNT/A is greater than 73 Units to 3000 unit, 100 Units to 2500 Units, more preferably 200 Units to 2000 Units.

As mentioned above, the unit dose of modified BoNT/A may be greater than 73 Units. An upper limit of the unit dose range may be 300, 250, 200, 150, 100 or 50 Units of modified BoNT/A, preferably the upper limit is 300 Units. A lower limit of the unit dose range may be 75, 100, 150, 200, 300 units, preferably the lower limit is 100 Units. Preferably, the unit dose of modified BoNT/A is 100-300 Units of modified BoNT/A, e.g. 150 Units to 250 Units.

The unit dose of modified BoNT/A may be selected from: 100 Units to 300 Units, 150 to 250 Units, and 175 Units to 225 Units.

The unit dose may be greater than 1754 pg to 8000 pg of modified BoNT/A. In other words, the unit dose may be greater than 1754 pg, and up to 8000 pg of modified BoNT/A. An upper limit of the unit dose range may be 8,000, 7,000, 6,000, 5,000, 4,000, 3,000, 2,000 or 1,000 pg of modified BoNT/A, preferably the upper limit is 5,000 pg. A lower limit of the unit dose range may be 1,800, 2,000, 2,500, 3000, or 4,000 pg of modified BoNT/A, preferably the lower limit is 1,800. The unit dose of modified BoNT/A may be selected from: 1,800 to 8,000 pg, 2,000 to 6,000 pg, most preferably 3,000 to 6,000 pg. The unit dose of modified BoNT/A may be 4,500 to 5,500 pg of modified BoNT/A, e.g. 4,900 to 5,100 pg.

The unit dose may be greater than 1800 pg of modified BoNT/A. The unit dose may be greater than 1800 pg and up to 7000 pg of modified BoNT/A, e.g. greater than 1800 pg and up to 6000 pg of modified BoNT/A. The unit dose may be greater than 2000 pg and up to 5000 pg of modified BoNT/A.

The unit dose may be greater than 2555 pg. For example, the unit dose may be greater than 2555 pg and up to 8000 pg of modified BoNT/A, for example greater than 2555 pg and up to 6000 pg.

The unit dose may be 2000 pg, 2500 pg, 3000 pg, 4000 pg, 4500 pg, 5000 pg, 5500 pg, 6000 pg, 6500 pg, 7000 pg, 7500 pg or 8000 pg of modified BoNT/A.

It is more preferred that the unit dose is 2000 to 3500 pg of modified BoNT/A. For example, the unit dose may be 2250 pg to 3250 pg of modified BoNT/A. A particularly preferred unit dose may be about 2500 pg (e.g. 2500 pg±10%) of modified BoNT/A, for example the unit dose may preferably be 2500 pg of modified BoNT/A. Another preferred unit dose may be about 3000 pg (e.g. 3000 pg±10%) of modified BoNT/A, for example the unit dose may be 3000 pg of modified BoNT/A.

The unit dose may be 3500-5500 pg of modified BoNT/A. For example, the unit dose may be 3800 pg to 4200 pg of modified BoNT/A. The unit dose may be 4800 to 5200 pg of modified BoNT/A. A particularly preferred unit dose may be about 4000 pg (e.g. 4000 pg±10%) of modified BoNT/A, for example the unit dose may be 4000 pg of modified BoNT/A. Another particularly preferred unit dose may be about 5000 pg (e.g. 5000 pg±10%) of modified BoNT/A, for example the unit dose may be 5000 pg of modified BoNT/A.

Unit doses can be expressed above in terms of “Units”, and also in terms of amounts in pg. A unit dose of modified BoNT/A may also be expressed in both Units and amounts (pg) simultaneously.

When treating facial lines, modified BoNT/A may be administered to a plurality of sites selected from: up to two sites of a corrugator muscle and one site of a procerus muscle for treating glabellar lines, up to five sites of a frontalis muscle for treating forehead lines; and up to three sites at the external part of a orbicularis oculi muscle for treating lateral canthal lines.

A total dose administered when carrying out the treatment regimen of the present invention may be up to 3661 Units. In other words, the total amount of modified BoNT/A administered at a given treatment session may be up to 3661 Units. The total dose may be up to 3500, 3000, 2500, 2000, 1500, or 1000 Units of modified BoNT/A. Preferably, the total dose may be up to 3327.8 Units of modified BoNT/A.

A total dose administered when carrying out the treatment regimen of the present invention may be up to 88000, 85000, 80000, 75000, 70000, 65000, 60000, 55000, 50000, 45000, 40000, 35000 or 30000 pg of modified BoNT/A, preferably up to 80000 pg of modified BoNT/A. Thus, an upper limit of the total dose may be 88000, 85000, 80000, 75000, 70000, 65000, 60000, 55000, 50000, 45000, 40000, 35000 or 30000 pg of modified BoNT/A, preferably 80000 pg of modified BoNT/A. A lower limit of the total dose may be 25000, 20000, 15000, 10000, 5000 pg, 2000 pg, 1000 pg, 500 pg, or 50 pg of modified BoNT/A. For example, a lower limit of the total dose may be 25000, 20000, 15000, 10000 or 5000 pg of modified BoNT/A.

A total dose administered when carrying out the treatment regimen of the present invention may be 7,000 pg to 8,000 pg of modified BoNT/A. Thus, 7,000 pg to 8,000 pg of modified BoNT/A may be administered to the subject (e.g. via a plurality of unit doses described herein). A particularly preferred total dose may be about 7,500 pg (e.g. 7,500 pg±10%) of modified BoNT/A, for example the total dose may be 7,500 pg of modified BoNT/A. Thus, about 7,500 pg (e.g. 7,500 pg±10%) of modified BoNT/A may be administered to the subject, for example 7,500 pg of modified BoNT/A may be administered to the subject (e.g. via a plurality of unit doses described herein).

A total dose administered when carrying out the treatment regimen of the present invention may be 10,000 pg to 20,000 pg of modified BoNT/A. Thus, 10,000 pg to 20,000 pg of modified BoNT/A may be administered to the subject (e.g. via a plurality of unit doses described herein). For example, the total dose may be 11,500 pg to 12,500 pg of modified BoNT/A. Thus, 11,500 pg to 12,500 pg of modified BoNT/A may be administered to the subject (e.g. via a plurality of unit doses described herein). A particularly preferred total dose may be about 12,000 pg (e.g. 12,000 pg±10%) of modified BoNT/A, for example the total dose may be 12,000 pg of modified BoNT/A. Thus, about 12,000 pg (e.g. 12,000 pg±10%) of modified BoNT/A may be administered to the subject, for example 12,000 pg of modified BoNT/A may be administered to the subject (e.g. via a plurality of unit doses described herein).

For example, the total dose may be 12,000 pg to 13,000 pg of modified BoNT/A. Thus, 12,000 pg to 13,000 pg of modified BoNT/A may be administered to the subject (e.g. via a plurality of unit doses described herein). A particularly preferred total dose may be about 12,500 pg (e.g. 12,500 pg±10%) of modified BoNT/A, for example the total dose may be 12,500 pg of modified BoNT/A. Thus, about 12,500 pg (e.g. 12,500 pg±10%) of modified BoNT/A may be administered to the subject, for example 12,500 pg of modified BoNT/A may be administered to the subject (e.g. via a plurality of unit doses described herein).

The total dose administered to the subject may be 14,000 pg to 16,000 pg of modified BoNT/A. Thus, 14,000 pg to 16,000 pg of modified BoNT/A may be administered to the subject (e.g. via a plurality of unit doses described herein). A particularly preferred total dose may be about 15,000 pg (e.g. 15,000 pg±10%) of modified BoNT/A, for example the total dose may be 15,000 pg of modified BoNT/A. Thus, about 15,000 pg (e.g. 15,000 pg±10%) of modified BoNT/A may be administered to the subject, for example 15,000 pg of modified BoNT/A may be administered to the subject (e.g. via a plurality of unit doses described herein).

A total dose administered when carrying out the treatment regimen of the present invention may be 18,000 pg to 26,000 pg of modified BoNT/A. Thus, 18,000 pg to 26,000 pg of modified BoNT/A may be administered to the subject (e.g. via a plurality of unit doses described herein). For example, the total dose may be 19,000 pg to 21,000 pg of modified BoNT/A. Thus, 19,000 pg to 21,000 pg of modified BoNT/A may be administered to the subject (e.g. via a plurality of unit doses described herein). A particularly preferred total dose may be about 20,000 pg (e.g. 20,000 pg±10%) of modified BoNT/A, for example the total dose may be 20,000 pg of modified BoNT/A. Thus, about 20,000 pg (e.g. 20,000 pg±10%) of modified BoNT/A may be administered to the subject, for example 20,000 pg of modified BoNT/A may be administered to the subject (e.g. via a plurality of unit doses described herein).

A total dose administered when carrying out the treatment regimen of the present invention may be 22,000 pg to 23,000 pg of modified BoNT/A. Thus, 22,000 pg to 23,000 pg of modified BoNT/A may be administered to the subject (e.g. via a plurality of unit doses described herein). A particularly preferred total dose may be about 22,500 pg (e.g. 22,500 pg±10%) of modified BoNT/A, for example the total dose may be 22,500 pg of modified BoNT/A. Thus, about 22,500 pg (e.g. 22,500 pg±10%) of modified BoNT/A may be administered to the subject, for example 22,500 pg of modified BoNT/A may be administered to the subject (e.g. via a plurality of unit doses described herein).

The total dose administered to the subject may be 23,000 pg to 25,000 pg of modified BoNT/A. Thus, 23,000 pg to 25,000 pg of modified BoNT/A may be administered to the subject (e.g. via a plurality of unit doses described herein). A particularly preferred total dose may be about 24,000 pg (e.g. 24,000 pg±10%) of modified BoNT/A, for example the total dose may be 24,000 pg of modified BoNT/A. Thus, about 24,000 pg (e.g. 24,000 pg±10%) of modified BoNT/A may be administered to the subject, for example 24,000 pg of modified BoNT/A may be administered to the subject (e.g. via a plurality of unit doses described herein).

The total dose administered to the subject may be 24,000 pg to 26,000 pg of modified BoNT/A. Thus, 24,000 pg to 26,000 pg of modified BoNT/A may be administered to the subject (e.g. via a plurality of unit doses described herein). A particularly preferred total dose may be about 25,000 pg (e.g. 24,000 pg±10%) of modified BoNT/A, for example the total dose may be 25,000 pg of modified BoNT/A. Thus, about 25,000 pg (e.g. 25,000 pg±10%) of modified BoNT/A may be administered to the subject, for example 25,000 pg of modified BoNT/A may be administered to the subject (e.g. via a plurality of unit doses described herein).

The total dose administered to the subject may be 27,000 pg to 28,000 pg of modified BoNT/A. Thus, 27,000 pg to 28,000 pg of modified BoNT/A may be administered to the subject (e.g. via a plurality of unit doses described herein). A particularly preferred total dose may be about 27,500 pg (e.g. 27,500 pg±10%) of modified BoNT/A, for example the total dose may be 27,500 pg of modified BoNT/A. Thus, about 27,500 pg (e.g. 27,500 pg±10%) of modified BoNT/A may be administered to the subject, for example 27,500 pg of modified BoNT/A may be administered to the subject (e.g. via a plurality of unit doses described herein).

The total dose administered to the subject may be 31,000 pg to 33,000 pg of modified BoNT/A. Thus, 31,000 pg to 33,000 pg of modified BoNT/A may be administered to the subject (e.g. via a plurality of unit doses described herein). A particularly preferred total dose may be about 32,000 pg (e.g. 32,000 pg±10%) of modified BoNT/A, for example the total dose may be 32,000 pg of modified BoNT/A. Thus, about 32,000 pg (e.g. 32,000 pg±10%) of modified BoNT/A may be administered to the subject, for example 32,000 pg of modified BoNT/A may be administered to the subject (e.g. via a plurality of unit doses described herein).

The total dose administered to the subject may be 35,000 pg to 37,000 pg of modified BoNT/A. Thus, 35,000 pg to 37,000 pg of modified BoNT/A may be administered to the subject (e.g. via a plurality of unit doses described herein). A particularly preferred total dose may be about 36,000 pg (e.g. 36,000 pg±10%) of modified BoNT/A, for example the total dose may be 36,000 pg of modified BoNT/A. Thus, about 36,000 pg (e.g. 36,000 pg±10%) of modified BoNT/A may be administered to the subject, for example 36,000 pg of modified BoNT/A may be administered to the subject (e.g. via a plurality of unit doses described herein).

A total dose administered when carrying out the treatment regimen of the present invention may be 35,000 to 45,000 pg of modified BoNT/A. Thus, 35,000 to 45,000 pg of modified BoNT/A may be administered to the subject (e.g. via a plurality of unit doses described herein). A particularly preferred total dose may be about 40,000 pg (e.g. 40,000 pg±10%) of modified BoNT/A, for example the total dose may be 40,000 pg of modified BoNT/A. Thus, about 40,000 pg (e.g. 40,000 pg±10%) of modified BoNT/A may be administered to the subject, for example 40,000 pg of modified BoNT/A may be administered to the subject (e.g. via a plurality of unit doses described herein).

A total dose administered when carrying out the treatment regimen of the present invention may be 43,000 to 44,000 pg of modified BoNT/A. Thus, 43,000 to 44,000 pg of modified BoNT/A may be administered to the subject (e.g. via a plurality of unit doses described herein). A particularly preferred total dose may be about 44,000 pg (e.g. 44,000 pg±10%) of modified BoNT/A, for example the total dose may be 44,000 pg of modified BoNT/A. Thus, about 44,000 pg (e.g. 44,000 pg±10%) of modified BoNT/A may be administered to the subject, for example 44,000 pg of modified BoNT/A may be administered to the subject (e.g. via a plurality of unit doses described herein).

A total dose administered when carrying out the treatment regimen of the present invention may be 60,000 to 70,000 pg of modified BoNT/A. Thus, 60,000 to 70,000 pg of modified BoNT/A may be administered to the subject (e.g. via a plurality of unit doses described herein). A preferred total dose administered when carrying out the treatment regimen of the present invention may be 63,000 to 65,000 pg of modified BoNT/A. Thus, 63,000 to 65,000 pg of modified BoNT/A may be administered to the subject (e.g. via a plurality of unit doses described herein). A particularly preferred total dose may be about 64,000 pg (e.g. 64,000 pg±10%) of modified BoNT/A, for example the total dose may be 64,000 pg of modified BoNT/A. Thus, about 64,000 pg (e.g. 64,000 pg±10%) of modified BoNT/A may be administered to the subject, for example 64,000 pg of modified BoNT/A may be administered to the subject (e.g. via a plurality of unit doses described herein).

The total dose may be 12,000 pg to 13,000 pg of modified BoNT/A and the unit dose may be 2000 to 3500 pg of modified BoNT/A. Thus, 12,000 pg to 13,000 pg of modified BoNT/A may be administered to the subject, wherein the unit dose is 2000 to 3500 pg of modified BoNT/A.

A particularly preferred total dose may be about 12,500 pg (e.g. 12,500 pg±10%) of modified BoNT/A, for example the total dose may be 12,500 pg of modified BoNT/A, wherein the unit dose is 2000 to 3500 pg of modified BoNT/A. A preferred total dose is 12,500 pg of modified BoNT/A, wherein the unit dose is 2000 to 3500 pg of modified BoNT/A. A more preferred total dose is 12,500 pg of modified BoNT/A, wherein the unit dose is 2500 pg of modified BoNT/A. Thus, 12,500 pg of modified BoNT/A may be administered to the subject, wherein the unit dose is 2500 pg of modified BoNT/A.

The total dose may be 14,000 pg to 16,000 pg of modified BoNT/A and the unit dose may be 2000 to 3500 pg of modified BoNT/A. Thus, 14,000 pg to 16,000 pg of modified BoNT/A may be administered to the subject, wherein the unit dose is 2000 to 3500 pg of modified BoNT/A. A particularly preferred total dose may be about 15,000 pg (e.g. 15,000 pg±10%) of modified BoNT/A, for example the total dose may be 15,000 pg of modified BoNT/A, wherein the unit dose is 2000 to 3500 pg of modified BoNT/A. A preferred total dose is 15,000 pg of modified BoNT/A, wherein the unit dose is 2000 to 3500 pg of modified BoNT/A. A more preferred total dose is 15,000 pg of modified BoNT/A, wherein the unit dose is 2500 pg of modified BoNT/A. Thus, 15,000 pg of modified BoNT/A may be administered to the subject, wherein the unit dose is 2500 pg of modified BoNT/A.

A total dose administered when carrying out the treatment regimen of the present invention may be 18,000 pg to 26,000 pg of modified BoNT/A, wherein the unit dose is 3500-5500 pg of modified BoNT/A. Thus, 18,000 pg to 26,000 pg of modified BoNT/A may be administered to the subject, wherein the unit dose is 3500-5500 pg of modified BoNT/A.

A particularly preferred total dose may be about 20,000 pg (e.g. 20,000 pg±10%) of modified BoNT/A, for example the total dose may be 20,000 pg of modified BoNT/A, wherein the unit dose is 3500-5500 pg of modified BoNT/A. A preferred total dose is 20,000 pg of modified BoNT/A, wherein the unit dose is 3500-5500 pg of modified BoNT/A. A more preferred total dose is 20,000 pg of modified BoNT/A, wherein the unit dose is 4000 pg of modified BoNT/A. Thus, 20,000 pg of modified BoNT/A may be administered to the subject, wherein the unit dose is 4000 pg of modified BoNT/A.

A total dose administered when carrying out the treatment regimen of the present invention may be 23,000 pg to 25,000 pg of modified BoNT/A, wherein the unit dose is 3500-5500 pg of modified BoNT/A. Thus, 23,000 pg to 25,000 pg of modified BoNT/A may be administered to the subject, wherein the unit dose is 3500-5500 pg of modified BoNT/A.

A particularly preferred total dose may be about 24,000 pg (e.g. 24,000 pg±10%) of modified BoNT/A, for example the total dose may be 24,000 pg of modified BoNT/A, wherein the unit dose is 3500-5500 pg of modified BoNT/A. A preferred total dose is 24,000 pg of modified BoNT/A, wherein the unit dose is 3500-5500 pg of modified BoNT/A. A more preferred total dose is 24,000 pg of modified BoNT/A, wherein the unit dose is 4000 pg of modified BoNT/A. Thus, 24,000 pg of modified BoNT/A may be administered to the subject, wherein the unit dose is 4000 pg of modified BoNT/A

A total dose administered when carrying out the treatment regimen of the present invention may be 43,000 to 44,000 pg of modified BoNT/A, wherein the unit dose is 2000 to 3500 pg of modified BoNT/A. Thus, 43,000 to 44,000 pg of modified BoNT/A may be administered to the subject, wherein the unit dose is 2000 to 3500 pg of modified BoNT/A. A particularly preferred total dose may be about 44,000 pg (e.g. 44,000 pg±10%) of modified BoNT/A wherein the unit dose is 2000 to 3500 pg of modified BoNT/A, for example the total dose may be 44,000 pg of modified BoNT/A wherein the unit dose is 2000 to 3500 pg of modified BoNT/A. Thus, about 44,000 pg (e.g. 44,000 pg±10%) of modified BoNT/A may be administered to the subject, wherein the unit dose is 2000 to 3500 pg of modified BoNT/A. It is preferred that 44,000 pg of modified BoNT/A may be administered to the subject, wherein the unit dose is 2000 to 3500 pg of modified BoNT/A. For example, about 44,000 pg (e.g. 44,000 pg±10%) of modified BoNT/A may be administered to the subject, wherein the unit dose is about 2500 pg (e.g. 2500 pg±10%) of modified BoNT/A. It is more preferred that 44,000 pg of modified BoNT/A may be administered to the subject, wherein the unit dose is 2500 pg of modified BoNT/A.

A total dose administered when carrying out the treatment regimen of the present invention may be 60,000 to 70,000 pg of modified BoNT/A, wherein the unit dose is 3500-5500 pg of modified BoNT/A. Thus, 60,000 to 70,000 pg of modified BoNT/A may be administered to the subject, wherein the unit dose is 3500-5500 pg of modified BoNT/A. A preferred total dose administered when carrying out the treatment regimen of the present invention may be 63,000 to 65,000 pg of modified BoNT/A, wherein the unit dose is 3500-5500 pg of modified BoNT/A. Thus, 63,000 to 65,000 pg of modified BoNT/A may be administered to the subject, wherein the unit dose is 3500-5500 pg of modified BoNT/A. A particularly preferred total dose may be about 64,000 pg (e.g. 64,000 pg±10%) of modified BoNT/A, wherein the unit dose is 3500-5500 pg of modified BoNT/A. For example the total dose may be 64,000 pg of modified BoNT/A, wherein the unit dose is 3500-5500 pg of modified BoNT/A. Thus, about 64,000 pg (e.g. 64,000 pg±10%) of modified BoNT/A may be administered to the subject, wherein the unit dose is 3500-5500 pg of modified BoNT/A. It is preferred that about 64,000 pg (e.g. 64,000 pg±10%) of modified BoNT/A may be administered to the subject, wherein the unit dose is about 4000 pg (e.g. 4,000 pg±10%) of modified BoNT/A. It is more preferred that 64,000 pg of modified BoNT/A may be administered to the subject, wherein the unit dose is 4000 pg of modified BoNT/A.

The total dose may vary depending on the facial line, or combination of facial lines to be treated. Examples of preferred total doses for any given facial line will be discussed in more detail below.

A total dose administered when carrying out the treatment regimen for glabellar lines may be up to 30000, 25000, 20000, 15000, 10000 or 5000 pg of modified BoNT/A, preferably up to 25000 pg of modified BoNT/A. A particularly preferred total dose that may be administered when carrying out the treatment regimen for glabellar lines may be about 12,500 pg (e.g. 12,500 pg±10%) of modified BoNT/A, for example the unit dose may be 12,500 pg of modified BoNT/A. Another particularly preferred total dose that may be administered when carrying out the treatment regimen for glabellar lines may be about 20,000 pg (e.g. 20,000 pg±10%) of modified BoNT/A, for example the unit dose may be 20,000 pg of modified BoNT/A.

A total dose administered when carrying out the treatment regimen for glabellar lines may be 7,000 pg to 8,000 pg of modified BoNT/A. Thus, 7,000 pg to 8,000 pg of modified BoNT/A may be administered to the subject (e.g. via a plurality of unit doses described herein) for treating glabellar lines. A particularly preferred total dose may be about 7,500 pg (e.g. 7,500 pg±10%) of modified BoNT/A for treating glabellar lines, for example the total dose may be 7,500 pg of modified BoNT/A for treating glabellar lines. Thus, about 7,500 pg (e.g. 7,500 pg±10%) of modified BoNT/A may be administered to the subject for treating glabellar lines, for example 7,500 pg of modified BoNT/A may be administered to the subject (e.g. via a plurality of unit doses described herein) for treating glabellar lines.

A total dose administered when carrying out the treatment regimen for glabellar lines may be 10,000 pg to 20,000 pg of modified BoNT/A. Thus, 10,000 pg to 20,000 pg of modified BoNT/A may be administered to the subject (e.g. via a plurality of unit doses described herein) for treating glabellar lines. For example, the total dose may be 11,500 pg to 12,500 pg of modified BoNT/A for treating glabellar lines. Thus, 11,500 pg to 12,500 pg of modified BoNT/A may be administered to the subject (e.g. via a plurality of unit doses described herein) for treating glabellar lines. A particularly preferred total dose may be about 12,000 pg (e.g. 12,000 pg±10%) of modified BoNT/A, for example the total dose may be 12,000 pg of modified BoNT/A for treating glabellar lines. Thus, about 12,000 pg (e.g. 12,000 pg±10%) of modified BoNT/A may be administered to the subject for treating glabellar lines, for example 12,000 pg of modified BoNT/A may be administered to the subject (e.g. via a plurality of unit doses described herein) for treating glabellar lines.

A total dose administered when carrying out the treatment regimen for glabellar lines may be 12,000 pg to 13,000 pg of modified BoNT/A. Thus, 12,000 pg to 13,000 pg of modified BoNT/A may be administered to the subject (e.g. via a plurality of unit doses described herein) for treating glabellar lines. A particularly preferred total dose may be about 12,500 pg (e.g. 12,500 pg±10%) of modified BoNT/A for treating glabellar lines, for example the total dose may be 12,500 pg of modified BoNT/A for treating glabellar lines. Thus, about 12,500 pg (e.g. 12,500 pg±10%) of modified BoNT/A may be administered to the subject for treating glabellar lines, for example 12,500 pg of modified BoNT/A may be administered to the subject (e.g. via a plurality of unit doses described herein) for treating glabellar lines.

A total dose administered when carrying out the treatment regimen for glabellar lines may be 18,000 pg to 26,000 pg of modified BoNT/A. Thus, 18,000 pg to 26,000 pg of modified BoNT/A may be administered to the subject (e.g. via a plurality of unit doses described herein) for treating glabellar lines. For example, the total dose may be 19,000 pg to 21,000 pg of modified BoNT/A for treating glabellar lines. Thus, 19,000 pg to 21,000 pg of modified BoNT/A may be administered to the subject (e.g. via a plurality of unit doses described herein) for treating glabellar lines. A particularly preferred total dose may be about 20,000 pg (e.g. 20,000 pg±10%) of modified BoNT/A for treating glabellar lines, for example the total dose may be 20,000 pg of modified BoNT/A for treating glabellar lines. Thus, about 20,000 pg (e.g. 20,000 pg±10%) of modified BoNT/A may be administered to the subject for treating glabellar lines, for example 20,000 pg of modified BoNT/A may be administered to the subject (e.g. via a plurality of unit doses described herein) for treating glabellar lines.

A total dose administered when carrying out the treatment regimen for forehead lines may be up to 30000, 25000, 20000, 15000, 10000 or 5000 pg of modified BoNT/A, preferably up to 25000 pg of modified BoNT/A.

A total dose administered when carrying out the treatment regimen for forehead lines may be 12,000 pg to 13,000 pg of modified BoNT/A. Thus, 12,000 pg to 13,000 pg of modified BoNT/A may be administered to the subject (e.g. via a plurality of unit doses described herein) for treating forehead lines. A particularly preferred total dose may be about 12,500 pg (e.g. 12,500 pg±10%) of modified BoNT/A for treating forehead lines, for example the total dose may be 12,500 pg of modified BoNT/A for treating forehead lines. Thus, about 12,500 pg (e.g. 12,500 pg±10%) of modified BoNT/A may be administered to the subject for treating forehead lines, for example 12,500 pg of modified BoNT/A may be administered to the subject (e.g. via a plurality of unit doses described herein) for treating forehead lines.

A total dose administered when carrying out the treatment regimen for treating forehead lines may be 18,000 pg to 26,000 pg of modified BoNT/A. Thus, 18,000 pg to 26,000 pg of modified BoNT/A may be administered to the subject (e.g. via a plurality of unit doses described herein) for treating forehead lines. For example, the total dose may be 19,000 pg to 21,000 pg of modified BoNT/A for treating forehead lines. Thus, 19,000 pg to 21,000 pg of modified BoNT/A may be administered to the subject (e.g. via a plurality of unit doses described herein) for treating forehead lines. A particularly preferred total dose may be about 20,000 pg (e.g. 20,000 pg±10%) of modified BoNT/A for treating forehead lines, for example the total dose may be 20,000 pg of modified BoNT/A for treating forehead lines. Thus, about 20,000 pg (e.g. 20,000 pg±10%) of modified BoNT/A may be administered to the subject for treating forehead lines, for example 20,000 pg of modified BoNT/A may be administered to the subject (e.g. via a plurality of unit doses described herein) for treating forehead lines.

A total dose administered when carrying out the treatment regimen for lateral canthal lines may be up to 30000, 25000, 20000, 15000, 10000 or 5000 pg of modified BoNT/A, preferably up to 25000 pg of modified BoNT/A.

A total dose administered when carrying out the treatment regimen for lateral canthal lines may be 7,000 pg to 8,000 pg of modified BoNT/A. Thus, 7,000 pg to 8,000 pg of modified BoNT/A may be administered to the subject (e.g. via a plurality of unit doses described herein) for treating lateral canthal lines. A particularly preferred total dose may be about 7,500 pg (e.g. 7,500 pg±10%) of modified BoNT/A for treating lateral canthal lines, for example the total dose may be 7,500 pg of modified BoNT/A for treating lateral canthal lines. Thus, about 7,500 pg (e.g. 7,500 pg±10%) of modified BoNT/A may be administered to the subject for treating lateral canthal lines, for example 7,500 pg of modified BoNT/A may be administered to the subject (e.g. via a plurality of unit doses described herein) for treating lateral canthal lines.

A total dose administered when carrying out the treatment regimen for lateral canthal lines may be 10,000 pg to 20,000 pg of modified BoNT/A. Thus, 10,000 pg to 20,000 pg of modified BoNT/A may be administered to the subject (e.g. via a plurality of unit doses described herein) for treating lateral canthal lines. For example, the total dose may be 11,500 pg to 12,500 pg of modified BoNT/A for treating lateral canthal lines. Thus, 11,500 pg to 12,500 pg of modified BoNT/A may be administered to the subject (e.g. via a plurality of unit doses described herein) for treating lateral canthal lines. A particularly preferred total dose may be about 12,000 pg (e.g. 12,000 pg±10%) of modified BoNT/A, for example the total dose may be 12,000 pg of modified BoNT/A for treating lateral canthal lines. Thus, about 12,000 pg (e.g. 12,000 pg±10%) of modified BoNT/A may be administered to the subject for treating lateral canthal lines, for example 12,000 pg of modified BoNT/A may be administered to the subject (e.g. via a plurality of unit doses described herein) for treating lateral canthal lines.

A total dose administered when carrying out the treatment regimen for lateral canthal lines may be 14,000 pg to 16,000 pg of modified BoNT/A. Thus, 14,000 pg to 16,000 pg of modified BoNT/A may be administered to the subject (e.g. via a plurality of unit doses described herein) for treating lateral canthal lines. A particularly preferred total dose may be about 15,000 pg (e.g. 15,000 pg±10%) of modified BoNT/A for treating lateral canthal lines, for example the total dose may be 15,000 pg of modified BoNT/A for treating lateral canthal lines. Thus, about 15,000 pg (e.g. 15,000 pg±10%) of modified BoNT/A may be administered to the subject for treating lateral canthal lines, for example 15,000 pg of modified BoNT/A may be administered to the subject (e.g. via a plurality of unit doses described herein) for treating lateral canthal lines.

A total dose administered when carrying out the treatment regimen for lateral canthal lines may be 23,000 pg to 25,000 pg of modified BoNT/A. Thus, 23,000 pg to 25,000 pg of modified BoNT/A may be administered to the subject (e.g. via a plurality of unit doses described herein) for treating lateral canthal lines. A particularly preferred total dose may be about 24,000 pg (e.g. 24,000 pg±10%) of modified BoNT/A for treating lateral canthal lines, for example the total dose may be 24,000 pg of modified BoNT/A for treating lateral canthal lines. Thus, about 24,000 pg (e.g. 24,000 pg±10%) of modified BoNT/A may be administered to the subject for treating lateral canthal lines, for example 24,000 pg of modified BoNT/A may be administered to the subject (e.g. via a plurality of unit doses described herein) for treating lateral canthal lines.

A total dose administered when carrying out the treatment regimen for glabellar lines and forehead lines may be 18,000 pg to 26,000 pg of modified BoNT/A. Thus, 18,000 pg to 26,000 pg of modified BoNT/A may be administered to the subject (e.g. via a plurality of unit doses described herein) for treating glabellar lines and forehead lines. For example, the total dose may be 19,000 pg to 21,000 pg of modified BoNT/A for treating glabellar lines and forehead lines. Thus, 19,000 pg to 21,000 pg of modified BoNT/A may be administered to the subject (e.g. via a plurality of unit doses described herein) for treating glabellar lines and forehead lines. A particularly preferred total dose may be about 20,000 pg (e.g. 20,000 pg±10%) of modified BoNT/A for treating glabellar lines and forehead lines, for example the total dose may be 20,000 pg of modified BoNT/A for treating glabellar lines and forehead lines. Thus, about 20,000 pg (e.g. 20,000 pg±10%) of modified BoNT/A may be administered to the subject for treating glabellar lines and forehead lines, for example 20,000 pg of modified BoNT/A may be administered to the subject (e.g. via a plurality of unit doses described herein) for treating glabellar lines and forehead lines.

A total dose administered when carrying out the treatment regimen for glabellar lines and forehead lines may be 24,000 pg to 26,000 pg of modified BoNT/A. Thus, 24,000 pg to 26,000 pg of modified BoNT/A may be administered to the subject (e.g. via a plurality of unit doses described herein) for treating glabellar lines and forehead lines. A particularly preferred total dose may be about 25,000 pg (e.g. 24,000 pg±10%) of modified BoNT/A for treating glabellar lines and forehead lines, for example the total dose may be 25,000 pg of modified BoNT/A for treating glabellar lines and forehead lines. Thus, about 25,000 pg (e.g. 25,000 pg±10%) of modified BoNT/A may be administered to the subject for treating glabellar lines and forehead lines, for example 25,000 pg of modified BoNT/A may be administered to the subject (e.g. via a plurality of unit doses described herein) for treating glabellar lines and forehead lines.

A total dose administered when carrying out the treatment regimen for glabellar lines and forehead lines may be 31,000 pg to 33,000 pg of modified BoNT/A. Thus, 31,000 pg to 33,000 pg of modified BoNT/A may be administered to the subject (e.g. via a plurality of unit doses described herein) for treating glabellar lines and forehead lines. A particularly preferred total dose may be about 32,000 pg (e.g. 32,000 pg±10%) of modified BoNT/A for treating glabellar lines and forehead lines, for example the total dose may be 32,000 pg of modified BoNT/A for treating glabellar lines and forehead lines. Thus, about 32,000 pg (e.g. 32,000 pg±10%) of modified BoNT/A may be administered to the subject for treating glabellar lines and forehead lines, for example 32,000 pg of modified BoNT/A may be administered to the subject (e.g. via a plurality of unit doses described herein) for treating glabellar lines and forehead lines.

A total dose administered when carrying out the treatment regimen for glabellar lines and forehead lines may be 35,000 to 45,000 pg of modified BoNT/A. Thus, 35,000 to 45,000 pg of modified BoNT/A may be administered to the subject (e.g. via a plurality of unit doses described herein) for treating glabellar lines and forehead lines. A particularly preferred total dose may be about 40,000 pg (e.g. 40,000 pg±10%) of modified BoNT/A for treating glabellar lines and forehead lines, for example the total dose may be 40,000 pg of modified BoNT/A for treating glabellar lines and forehead lines. Thus, about 40,000 pg (e.g. 40,000 pg±10%) of modified BoNT/A may be administered to the subject for treating glabellar lines and forehead lines, for example 40,000 pg of modified BoNT/A may be administered to the subject (e.g. via a plurality of unit doses described herein) for treating glabellar lines and forehead lines.

A total dose administered when carrying out the treatment regimen for glabellar lines and lateral canthal lines may be 14,000 pg to 16,000 pg of modified BoNT/A. Thus, 14,000 pg to 16,000 pg of modified BoNT/A may be administered to the subject (e.g. via a plurality of unit doses described herein) for treating glabellar lines and lateral canthal lines. A particularly preferred total dose may be about 15,000 pg (e.g. 15,000 pg±10%) of modified BoNT/A for treating glabellar lines and lateral canthal lines, for example the total dose may be 15,000 pg of modified BoNT/A for treating glabellar lines and lateral canthal lines. Thus, about 15,000 pg (e.g. 15,000 pg±10%) of modified BoNT/A may be administered to the subject for treating glabellar lines and lateral canthal lines, for example 15,000 pg of modified BoNT/A may be administered to the subject (e.g. via a plurality of unit doses described herein) for treating glabellar lines and lateral canthal lines.

A total dose administered when carrying out the treatment regimen for glabellar lines and lateral canthal lines may be 18,000 pg to 26,000 pg of modified BoNT/A. Thus, 18,000 pg to 26,000 pg of modified BoNT/A may be administered to the subject (e.g. via a plurality of unit doses described herein) for treating glabellar lines and lateral canthal lines. For example, the total dose may be 19,000 pg to 21,000 pg of modified BoNT/A for treating glabellar lines and lateral canthal lines. Thus, 19,000 pg to 21,000 pg of modified BoNT/A may be administered to the subject (e.g. via a plurality of unit doses described herein) for treating glabellar lines and lateral canthal lines. A particularly preferred total dose may be about 20,000 pg (e.g. 20,000 pg±10%) of modified BoNT/A for treating glabellar lines and lateral canthal lines, for example the total dose may be 20,000 pg of modified BoNT/A for treating glabellar lines and lateral canthal lines. Thus, about 20,000 pg (e.g. 20,000 pg±10%) of modified BoNT/A may be administered to the subject for treating glabellar lines and lateral canthal lines, for example 20,000 pg of modified BoNT/A may be administered to the subject (e.g. via a plurality of unit doses described herein) for treating glabellar lines and lateral canthal lines.

A total dose administered when carrying out the treatment regimen for glabellar lines and lateral canthal lines may be 22,000 pg to 23,000 pg of modified BoNT/A. Thus, 22,000 pg to 23,000 pg of modified BoNT/A may be administered to the subject (e.g. via a plurality of unit doses described herein) for treating glabellar lines and lateral canthal lines. A particularly preferred total dose may be about 22,500 pg (e.g. 22,500 pg±10%) of modified BoNT/A for treating glabellar lines and lateral canthal lines, for example the total dose may be 22,500 pg of modified BoNT/A for treating glabellar lines and lateral canthal lines. Thus, about 22,500 pg (e.g. 22,500 pg±10%) of modified BoNT/A may be administered to the subject for treating glabellar lines and lateral canthal lines, for example 22,500 pg of modified BoNT/A may be administered to the subject (e.g. via a plurality of unit doses described herein) for treating glabellar lines and lateral canthal lines.

A total dose administered when carrying out the treatment regimen for glabellar lines and lateral canthal lines may be 23,000 pg to 25,000 pg of modified BoNT/A. Thus, 23,000 pg to 25,000 pg of modified BoNT/A may be administered to the subject (e.g. via a plurality of unit doses described herein) for treating glabellar lines and lateral canthal lines. A particularly preferred total dose may be about 24,000 pg (e.g. 24,000 pg±10%) of modified BoNT/A for treating glabellar lines and lateral canthal lines, for example the total dose may be 24,000 pg of modified BoNT/A for treating glabellar lines and lateral canthal lines. Thus, about 24,000 pg (e.g. 24,000 pg±10%) of modified BoNT/A may be administered to the subject for treating glabellar lines and lateral canthal lines, for example 24,000 pg of modified BoNT/A may be administered to the subject (e.g. via a plurality of unit doses described herein) for treating lateral canthal lines.

A total dose administered when carrying out the treatment regimen for glabellar lines and lateral canthal lines may be 27,000 pg to 28,000 pg of modified BoNT/A. Thus, 27,000 pg to 28,000 pg of modified BoNT/A may be administered to the subject (e.g. via a plurality of unit doses described herein) for treating glabellar lines and lateral canthal lines. A particularly preferred total dose may be about 27,500 pg (e.g. 27,500 pg±10%) of modified BoNT/A for treating glabellar lines and lateral canthal lines, for example the total dose may be 27,500 pg of modified BoNT/A for treating glabellar lines and lateral canthal lines. Thus, about 27,500 pg (e.g. 27,500 pg±10%) of modified BoNT/A may be administered to the subject for treating glabellar lines and lateral canthal lines, for example 27,500 pg of modified BoNT/A may be administered to the subject (e.g. via a plurality of unit doses described herein) for treating glabellar lines and lateral canthal lines.

A total dose administered when carrying out the treatment regimen for glabellar lines and lateral canthal lines may be 31,000 pg to 33,000 pg of modified BoNT/A. Thus, 31,000 pg to 33,000 pg of modified BoNT/A may be administered to the subject (e.g. via a plurality of unit doses described herein) for treating glabellar lines and lateral canthal lines. A particularly preferred total dose may be about 32,000 pg (e.g. 32,000 pg±10%) of modified BoNT/A for treating glabellar lines and lateral canthal lines, for example the total dose may be 32,000 pg of modified BoNT/A for treating glabellar lines and lateral canthal lines. Thus, about 32,000 pg (e.g. 32,000 pg±10%) of modified BoNT/A may be administered to the subject for treating glabellar lines and lateral canthal lines, for example 32,000 pg of modified BoNT/A may be administered to the subject (e.g. via a plurality of unit doses described herein) for treating glabellar lines and lateral canthal lines.

A total dose administered when carrying out the treatment regimen for glabellar lines and lateral canthal lines may be 35,000 pg to 37,000 pg of modified BoNT/A. Thus, 35,000 pg to 37,000 pg of modified BoNT/A may be administered to the subject (e.g. via a plurality of unit doses described herein) for treating glabellar lines and lateral canthal lines. A particularly preferred total dose may be about 36,000 pg (e.g. 36,000 pg±10%) of modified BoNT/A for treating glabellar lines and lateral canthal lines, for example the total dose may be 36,000 pg of modified BoNT/A for treating glabellar lines and lateral canthal lines. Thus, about 36,000 pg (e.g. 36,000 pg±10%) of modified BoNT/A may be administered to the subject for treating glabellar lines and lateral canthal lines, for example 36,000 pg of modified BoNT/A may be administered to the subject (e.g. via a plurality of unit doses described herein) for treating glabellar lines and lateral canthal lines.

A total dose administered when carrying out the treatment regimen for glabellar lines and lateral canthal lines may be 43,000 to 44,000 pg of modified BoNT/A. Thus, 43,000 to 44,000 pg of modified BoNT/A may be administered to the subject (e.g. via a plurality of unit doses described herein) for treating glabellar lines and lateral canthal lines. A particularly preferred total dose may be about 44,000 pg (e.g. 44,000 pg±10%) of modified BoNT/A for treating glabellar lines and lateral canthal lines, for example the total dose may be 44,000 pg of modified BoNT/A for treating glabellar lines and lateral canthal lines. Thus, about 44,000 pg (e.g. 44,000 pg±10%) of modified BoNT/A may be administered to the subject for treating glabellar lines and lateral canthal lines, for example 44,000 pg of modified BoNT/A may be administered to the subject (e.g. via a plurality of unit doses described herein) for treating glabellar lines and lateral canthal lines.

A total dose administered when carrying out the treatment regimen for forehead lines and lateral canthal lines may be 18,000 pg to 26,000 pg of modified BoNT/A. Thus, 18,000 pg to 26,000 pg of modified BoNT/A may be administered to the subject (e.g. via a plurality of unit doses described herein) for treating forehead lines and lateral canthal lines. For example, the total dose may be 19,000 pg to 21,000 pg of modified BoNT/A for treating forehead lines and lateral canthal lines. Thus, 19,000 pg to 21,000 pg of modified BoNT/A may be administered to the subject (e.g. via a plurality of unit doses described herein) for treating forehead lines and lateral canthal lines. A particularly preferred total dose may be about 20,000 pg (e.g. 20,000 pg±10%) of modified BoNT/A for treating forehead lines and lateral canthal lines, for example the total dose may be 20,000 pg of modified BoNT/A for treating forehead lines and lateral canthal lines. Thus, about 20,000 pg (e.g. 20,000 pg±10%) of modified BoNT/A may be administered to the subject for treating forehead lines and lateral canthal lines, for example 20,000 pg of modified BoNT/A may be administered to the subject (e.g. via a plurality of unit doses described herein) for treating forehead lines and lateral canthal lines.

A total dose administered when carrying out the treatment regimen for forehead lines and lateral canthal lines may be 27,000 pg to 28,000 pg of modified BoNT/A. Thus, 27,000 pg to 28,000 pg of modified BoNT/A may be administered to the subject (e.g. via a plurality of unit doses described herein) for treating forehead lines and lateral canthal lines. A particularly preferred total dose may be about 27,500 pg (e.g. 27,500 pg±10%) of modified BoNT/A for treating forehead lines and lateral canthal lines, for example the total dose may be 27,500 pg of modified BoNT/A for treating forehead lines and lateral canthal lines. Thus, about 27,500 pg (e.g. 27,500 pg±10%) of modified BoNT/A may be administered to the subject for treating forehead lines and lateral canthal lines, for example 27,500 pg of modified BoNT/A may be administered to the subject (e.g. via a plurality of unit doses described herein) for treating forehead lines and lateral canthal lines.

A total dose administered when carrying out the treatment regimen for forehead lines and lateral canthal lines may be 31,000 pg to 33,000 pg of modified BoNT/A. Thus, 31,000 pg to 33,000 pg of modified BoNT/A may be administered to the subject (e.g. via a plurality of unit doses described herein) for treating forehead lines and lateral canthal lines. A particularly preferred total dose may be about 32,000 pg (e.g. 32,000 pg±10%) of modified BoNT/A for treating forehead lines and lateral canthal lines, for example the total dose may be 32,000 pg of modified BoNT/A for treating forehead lines and lateral canthal lines. Thus, about 32,000 pg (e.g. 32,000 pg±10%) of modified BoNT/A may be administered to the subject for treating forehead lines and lateral canthal lines, for example 32,000 pg of modified BoNT/A may be administered to the subject (e.g. via a plurality of unit doses described herein) for treating forehead lines and lateral canthal lines.

A total dose administered when carrying out the treatment regimen for forehead lines and lateral canthal lines may be 43,000 to 44,000 pg of modified BoNT/A. Thus, 43,000 to 44,000 pg of modified BoNT/A may be administered to the subject (e.g. via a plurality of unit doses described herein) for treating forehead lines and lateral canthal lines. A particularly preferred total dose may be about 44,000 pg (e.g. 44,000 pg±10%) of modified BoNT/A for treating forehead lines and lateral canthal lines, for example the total dose may be 44,000 pg of modified BoNT/A for treating forehead lines and lateral canthal lines. Thus, about 44,000 pg (e.g. 44,000 pg±10%) of modified BoNT/A may be administered to the subject for treating forehead lines and lateral canthal lines, for example 44,000 pg of modified BoNT/A may be administered to the subject (e.g. via a plurality of unit doses described herein) for treating forehead lines and lateral canthal lines.

A total dose administered when carrying out the treatment regimen for glabellar lines, forehead lines and lateral canthal lines may be 43,000 to 44,000 pg of modified BoNT/A. Thus, 43,000 to 44,000 pg of modified BoNT/A may be administered to the subject (e.g. via a plurality of unit doses described herein) for treating glabellar lines, forehead lines and lateral canthal lines. A particularly preferred total dose may be about 44,000 pg (e.g. 44,000 pg±10%) of modified BoNT/A for treating glabellar lines, forehead lines and lateral canthal lines, for example the total dose may be 44,000 pg of modified BoNT/A for treating glabellar lines, forehead lines and lateral canthal lines. Thus, about 44,000 pg (e.g. 44,000 pg±10%) of modified BoNT/A may be administered to the subject for treating glabellar lines, forehead lines and lateral canthal lines, for example 44,000 pg of modified BoNT/A may be administered to the subject (e.g. via a plurality of unit doses described herein) for treating glabellar lines, forehead lines and lateral canthal lines.

A total dose administered when carrying out the treatment regimen for glabellar lines, forehead lines and lateral canthal lines may be 60,000 to 70,000 pg of modified BoNT/A. Thus, 60,000 to 70,000 pg of modified BoNT/A may be administered to the subject (e.g. via a plurality of unit doses described herein) for treating glabellar lines, forehead lines and lateral canthal lines. A preferred total dose administered when carrying out the treatment regimen for glabellar lines, forehead lines and lateral canthal lines may be 63,000 to 65,000 pg of modified BoNT/A. Thus, 63,000 to 65,000 pg of modified BoNT/A may be administered to the subject (e.g. via a plurality of unit doses described herein) for treating glabellar lines, forehead lines and lateral canthal lines. A particularly preferred total dose may be about 64,000 pg (e.g. 64,000 pg±10%) of modified BoNT/A for treating glabellar lines, forehead lines and lateral canthal lines, for example the total dose may be 64,000 pg of modified BoNT/A for treating glabellar lines, forehead lines and lateral canthal lines. Thus, about 64,000 pg (e.g. 64,000 pg±10%) of modified BoNT/A may be administered to the subject for treating glabellar lines, forehead lines and lateral canthal lines, for example 64,000 pg of modified BoNT/A may be administered to the subject (e.g. via a plurality of unit doses described herein) for treating glabellar lines, forehead lines and lateral canthal lines.

A total dose administered when carrying out the treatment regimen for glabellar lines, forehead lines and lateral canthal lines may be 75,000 pg to 88,000 pg of modified BoNT/A. Thus, 75,000 pg to 88,000 pg of modified BoNT/A may be administered to the subject (e.g. via a plurality of unit doses described herein) for treating glabellar lines, forehead lines and lateral canthal lines. A particularly preferred total dose may be about 80,000 pg (e.g. 80,000 pg±10%) of modified BoNT/A for treating glabellar lines, forehead lines and lateral canthal lines, for example the total dose may be 80,000 pg of modified BoNT/A for treating glabellar lines, forehead lines and lateral canthal lines. Thus, about 80,000 pg (e.g. 80,000 pg±10%) of modified BoNT/A may be administered to the subject for treating glabellar lines, forehead lines and lateral canthal lines, for example 80,000 pg of modified BoNT/A may be administered to the subject (e.g. via a plurality of unit doses described herein) for treating glabellar lines, forehead lines and lateral canthal lines.

Where each of glabellar lines, forehead lines and lateral canthal lines are treated, the total doses may be up 88,000 pg (e.g. up to 80,000 pg).

The skilled person will take into consideration when a subject has recently had (or is subsequently having) additional treatment with a clostridial neurotoxin (e.g. BoNT), e.g. as part of a cosmetic treatment or treatment for a different indication. Using techniques routine in the art, the skilled person will adapt the present treatment regimen accordingly.

Administration to the plurality of muscles in accordance with the present invention preferably occurs in the same treatment session.

A modified BoNT/A of the invention preferably has a longer duration of action when compared to unmodified BoNT/A (e.g. Dysport®). The term “treating facial lines for a longer duration than that treated by an unmodified BoNT/A” may mean that the facial lines are reduced for a longer time period following administration of a modified BoNT/A of the invention, when compared to administration of an unmodified BoNT/A. Said duration of action may be at least 1.25×, 1.5×, 1.75×, 2.0×, or 2.25× greater. The duration of action of modified BoNT/A may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months. In particular embodiments, a duration of action may be greater than 9.0 months. Preferably, a duration of action may be greater than 9.0 months. Said reduction may be determined by comparison to an equivalent control subject exhibiting equivalent symptoms that has been treated with an unmodified BoNT/A. At a time period where the severity of one or more symptoms of the control subject are substantially the same (e.g. the same) as before unmodified BoNT/A treatment, a subject treated with a modified BoNT/A according to the invention may exhibit an improvement in the equivalent one or more symptoms of at least 5%, 10%, 25%, or 50% when compared to the severity of the one or more symptoms before treatment with the modified BoNT/A. The unmodified BoNT/A is preferably SEQ ID NO: 2 present in a di-chain form. In other words, the unmodified BoNT/A described herein is preferably a di-chain form of SEQ ID NO: 2.

Treatment may be repeated at an appropriate time period following administration of modified BoNT/A. Given that the duration of action is approximately twice that of unmodified BoNT/A (e.g. Dysport®) there are suitably longer periods between subsequent administrations than when a subject is treated with unmodified BoNT/A (e.g. Dysport®). A subject may be re-administered a modified BoNT/A in accordance with the present invention at least 18, 20, 25 or 30 weeks following a previous administration. For example, a subject may be re-administered a modified BoNT/A in accordance with the present invention at least 18-45 weeks, preferably 20-35 weeks following a previous administration.

A “subject” as used herein may be a mammal, such as a human or other mammal. Preferably “subject” means a human subject. The subject is preferably a subject that has no disease state associated with the muscle to which the modified BoNT/A is administered. For example, it is preferable that the subject does not have blepharospasm or hemifacial spasm (e.g. typical hemifacial spasm). It is particularly preferred that the subject does not have blepharospasm.

A subject for treatment in accordance with the invention may be a subject that is unsuitable for treatment with an unmodified BoNT/A and/or with another clostridial neurotoxin. Said subject may be a subject that is resistant to treatment with an unmodified BoNT/A and/or with another clostridial neurotoxin. Resistance may arise due to development of an immune response to a clostridial neurotoxin, including production of anti-clostridial neurotoxin antibodies, by a subject.

The term “treat” or “treating” as used herein refers to cosmetic treatment of facial lines, and encompasses prophylactic treatment (e.g. to prevent onset of a facial lines) as well as corrective treatment (treatment of a subject already having facial lines). Thus, any method, modified BoNT/A for use, or use described herein may suitably be described as a “cosmetic method for treating said facial line with said modified BoNT/A”. Additionally or alternatively, any method, modified BoNT/A for use, or use described herein may suitably be described as a “non-therapeutic use of said modified BoNT/A for treating said facial line”.

Preferably “treat” or “treating” as used herein means corrective treatment. The term “treat” or “treating” as used herein refers to the facial line and/or a symptom thereof. A treatment described herein may provide for a reduction in the level of glabellar lines by at least 5%, 10%, 25%, or 50% relative to the level of glabellar lines pre-treatment. A treatment described herein may provide for a reduction in the level of forehead lines by at least 5%, 10%, 25%, or 50% relative to the level of forehead lines pre-treatment. A treatment described herein may provide for a reduction in the level of lateral canthal lines by at least 5%, 10%, 25%, or 50% relative to the level of lateral canthal lines pre-treatment. A treatment described herein may provide for a reduction in the level of glabellar lines and lateral canthal lines by at least 5%, 10%, 25%, or 50% relative to the level of glabellar lines and lateral canthal lines pre-treatment. A treatment described herein may provide for a reduction in the level of glabellar lines and forehead lines by at least 5%, 10%, 25%, or 50% relative to the level of glabellar lines and forehead lines pre-treatment. A treatment described herein may provide for a reduction in the level of lateral canthal lines and forehead lines by at least 5%, 10%, 25%, or 50% relative to the level of lateral canthal lines and forehead lines pre-treatment. A treatment described herein may provide for a reduction in the level of glabellar lines, lateral canthal lines and forehead lines by at least 5%, 10%, 25%, or 50% relative to the level of glabellar lines, lateral canthal lines and forehead lines pre-treatment.

BoNT/A is one example of a clostridial neurotoxin produced by bacteria in the genus Clostridia. Other examples of such clostridial neurotoxins include those produced by C. tetani (TeNT) and by C. botulinum (BoNT) serotypes B-G and X (see WO 2018/009903 A2), as well as those produced by C. baratii and C. butyricum. Said neurotoxins are highly potent and specific and can poison neurons and other cells to which they are delivered. The clostridial toxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins (including BoNT/A) are synthesised as a single-chain polypeptide that is modified post-translationally by a proteolytic cleavage event to form two polypeptide chains joined together by a disulphide bond. Cleavage occurs at a specific cleavage site, often referred to as the activation site (e.g. activation loop), that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial toxins are non-cytotoxic proteases that cleave SNARE proteins.

In view of the ubiquitous nature of SNARE proteins, clostridial neurotoxins such as botulinum toxin have been successfully employed in a wide range of therapies.

For further details on the genetic basis of toxin production in Clostridium botulinum and C. tetani, see Henderson et al (1997) in The Clostridia: Molecular Biology and Pathogenesis, Academic press.

As discussed above, clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

Clostridial neurotoxin domains are described in more detail below.

Examples of L-chain reference sequences include:
Botulinum type A neurotoxin: amino acid residues 1-448
Botulinum type B neurotoxin: amino acid residues 1-440

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441

The translocation domain is a fragment of the H-chain of a clostridial neurotoxin approximately equivalent to the amino-terminal half of the H-chain, or the domain corresponding to that fragment in the intact H-chain.

Examples of reference translocation domains include:
Botulinum type A neurotoxin—amino acid residues (449-871)
Botulinum type B neurotoxin—amino acid residues (441-858)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)

In the context of the present invention, a variety of BoNT/A H_{N} regions comprising a translocation domain can be useful in aspects of the present invention. The H_{N} regions from the heavy-chain of BoNT/A are approximately 410-430 amino acids in length and comprise a translocation domain. Research has shown that the entire length of a H_{N} region from a clostridial neurotoxin heavy-chain is not necessary for the translocating activity of the translocation domain. Thus, aspects of this embodiment can include BoNT/A H_{N} regions comprising a translocation domain having a length of, for example, at least 350 amino acids, at least 375 amino acids, at least 400 amino acids or at least 425 amino acids. Other aspects of this embodiment can include BoNT/A H_{N} regions comprising a translocation domain having a length of, for example, at most 350 amino acids, at most 375 amino acids, at most 400 amino acids or at most 425 amino acids.

The term H_{N} embraces naturally-occurring BoNT/A H_{N} portions, and modified BoNT/A H_{N} portions having amino acid sequences that do not occur in nature and/or synthetic amino acid residues. Preferably, said modified BoNT/A H_{N} portions still demonstrate the above-mentioned translocation function.

Examples of clostridial neurotoxin receptor binding domain (H_{C}) reference sequences include:
BoNT/A—N872-L1296
BoNT/B—E859-E1291

The ˜50 kDa H_{C} domain of a clostridial neurotoxin (such as a BoNT) comprises two distinct structural features that are referred to as the H_{CC} and H_{CN} domains, each typically of ˜25 kDa. Amino acid residues involved in receptor binding are believed to be primarily located in the H_{CC} domain. The H_{C} domain of a native clostridial neurotoxin may comprise approximately 400-440 amino acid residues. This fact is confirmed by the following publications, each of which is herein incorporated in its entirety by reference thereto: Umland T C (1997) Nat. Struct. Biol. 4:788-792; Herreros J (2000) Biochem. J. 347:199-204; Halpern J (1993) J. Biol. Chem. 268:15, pp. 11188-11192; Rummel A (2007) PNAS 104:359-364; Lacey D B (1998) Nat. Struct. Biol. 5:898-902; Knapp (1998) Am. Cryst. Assoc. Abstract Papers 25:90; Swaminathan and Eswaramoorthy (2000) Nat. Struct. Biol. 7:1751-1759; and Rummel A (2004) Mol. Microbiol. 51(3), 631-643.

Examples of (reference) H_{CN} domains include:
Botulinum type A neurotoxin—amino acid residues (872-1110)
Botulinum type B neurotoxin—amino acid residues (859-1097)

The above sequence positions may vary a little according to serotype/sub-type, and further examples of (reference) H_{CN} domains include:
Botulinum type A neurotoxin—amino acid residues (874-1110)
Botulinum type B neurotoxin—amino acid residues (861-1097)

Examples of (reference) H_{CC} domains include:
Botulinum type A neurotoxin—amino acid residues (Y1111-L1296)
Botulinum type B neurotoxin—amino acid residues (Y1098-E1291)

WO 2017/191315 A1 (which is incorporated herein by reference) teaches modified BoNT/A (e.g. a “chimeric clostridial neurotoxins”) and methods for preparing and manufacturing the same. Thus, a modified BoNT/A comprising a botulinum neurotoxin A (BoNT/A) light-chain and translocation domain (BoNT/A H_{N}), and a BoNT/B receptor binding domain (H_{C} domain) for use in the present invention may be one taught in WO 2017/191315 A1.

The modified BoNT/A may be referred to as a “chimeric clostridial neurotoxin”. The term “chimeric clostridial neurotoxin” or “chimeric neurotoxin” as used herein means a neurotoxin comprising (preferably consisting of) a clostridial neurotoxin light-chain and translocation domain (H_{N} domain) from a first clostridial neurotoxin serotype and a receptor binding domain (H_{C} domain) originating from a second different clostridial neurotoxin serotype.

Specifically, a modified BoNT/A (e.g. chimeric clostridial neurotoxin) for use in the invention comprises a botulinum neurotoxin A (BoNT/A) light-chain and translocation domain (H_{N} domain), and a BoNT/B receptor binding domain (H_{C} domain). The BoNT/A LH_{N} domain of the modified BoNT/A (e.g. chimeric clostridial neurotoxin) is covalently linked to the BoNT/B H_{C} domain. The modified BoNT/A (e.g. chimeric clostridial neurotoxin) of the invention may be referred to as a chimeric botulinum neurotoxin. Said chimeric clostridial neurotoxin is also referred to herein as “BoNT/AB”, “mrBoNT/AB” or a “BoNT/AB chimera”.

The L-chain and H_{N} domain (optionally including a complete or partial activation loop, e.g. a complete activation loop when the modified BoNT/A is in a single-chain form and a cleaved/partial activation loop when in a di-chain form) may be collectively referred to as an LH_{N} domain. The LH_{N} domain thus does not further comprise an H_{C} domain.

The modified BoNT/A may consist essentially of a botulinum neurotoxin A (BoNT/A) light-chain and translocation domain (H_{N} domain), and a BoNT/B receptor binding domain (H_{C} domain).

The term “consist(s) essentially of” as used in this context means that the modified BoNT/A does not further comprise one or more amino acid residues that confer additional functionality to the polypeptide, e.g. when administered to a subject. In other words, a polypeptide that “consists essentially of” a botulinum neurotoxin A (BoNT/A) light-chain and translocation domain (H_{N} domain), and a BoNT/B receptor binding domain (H_{C} domain) may further comprise one or more amino acid residues (to those of the botulinum neurotoxin A (BoNT/A) light-chain and translocation domain (H_{N} domain), and BoNT/B receptor binding domain (H_{C} domain)) but said one or more further amino acid residues do not confer additional functionality to the polypeptide, e.g. when administered to a subject. Additional functionality may include enzymatic activity, binding activity and/or any physiological activity whatsoever.

The modified BoNT/A may comprise non-clostridial neurotoxin sequences in addition to any clostridial neurotoxin sequences so long as the non-clostridial neurotoxin sequences do not disrupt the ability of the modified BoNT/A (e.g. chimeric clostridial neurotoxin) to achieve its therapeutic effect. Preferably, the non-clostridial neurotoxin sequence is not one having catalytic activity, e.g. enzymatic activity. In one embodiment the modified BoNT/A of the invention does not comprise a non-clostridial catalytically active domain. In one embodiment, a modified BoNT/A does not comprise a further catalytically active domain. In one embodiment, the non-clostridial sequence is not one that binds to a cellular receptor. In other words, in one embodiment, the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp#db_reports. Non-clostridial neurotoxin sequences may include tags to aid in purification, such as His-tags. In one embodiment, a modified BoNT/A of the invention does not comprise a label or a site for adding a label, such as a sortase acceptor or donor site.

Preferably, a modified BoNT/A may consist of a botulinum neurotoxin A (BoNT/A) light-chain and translocation domain (H_{N} domain), and a BoNT/B receptor binding domain (H_{C} domain).

The modified BoNT/A comprises a light-chain that is capable of exhibiting non-cytotoxic protease activity and of cleaving a SNARE protein in the cytosol of a target neuron. Cell-based and in vivo assays may be used to determine if a clostridial neurotoxin comprising an L-chain and a functional cell binding and translocation domain has non-cytotoxic protease activity. Assays such as the Digit Abduction Score (DAS) assay, the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Aoki K R, Toxicon 39:1815-1820; 2001 or Donald et al (2018), Pharmacol Res Perspect, e00446, 1-14, which are incorporated herein by reference.

When administered to a subject, a modified BoNT/A is preferably in its active di-chain form where the light-chain and heavy-chain are joined together by a disulphide bond. Where a BoNT/A (e.g. modified BoNT/A) is defined herein by way of a polypeptide sequence (SEQ ID NO), an L-chain portion of the sequence (SEQ ID NO) may constitute a first chain of the di-chain clostridial neurotoxin (e.g. di-chain modified BoNT/A) and the H_{N} and H_{C} domains together may constitute a second chain of the di-chain clostridial neurotoxin (e.g. di-chain modified BoNT/A), wherein the first and second chains are joined together by a di-sulphide bond. The skilled person will appreciate that a protease may cleave at one or more positions within the activation loop of the clostridial neurotoxin (e.g. modified BoNT/A), preferably at two positions within the activation loop. Where cleavage occurs at more than one position (preferably at two positions) within the activation loop, a small fragment of the C-terminal L-chain portion of the sequence may be absent from the di-chain clostridial neurotoxin sequence (e.g. di-chain modified BoNT/A). In view of this, the sequence of the di-chain clostridial neurotoxin (e.g. di-chain modified BoNT/A) may be slightly different to that of the corresponding single-chain clostridial neurotoxin (e.g. single-chain modified BoNT/A). The small fragment may be 1-15 amino acids. In particular, in one embodiment, when Lys-C is used to covert a single-chain modified BoNT/A into a di-chain modified BoNT/A, the small fragment of the C-terminal L-chain portion of the sequence that is absent may be SEQ ID NO: 9 or 10.

Most preferably, a modified BoNT/A for use in the invention may comprise a BoNT/A light-chain and translocation domain (a BoNT/A LH_{N} domain), and a BoNT/B H_{C} domain. The BoNT/A LH_{N} domain is covalently linked to the BoNT/B H_{C} domain. Said modified BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

An example of a BoNT/A polypeptide sequence is provided as SEQ ID NO: 2.

An example of a BoNT/B polypeptide sequence is provided as SEQ ID NO: 8 (UniProt accession number B1INP5).

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureId=3BTA) and 1EPW (http://www.rcsb.org/pdb/explore/explore.do?structureId=1EPW) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular, the region around the “H_{N}/H_{CN}” junction may be structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) may be used to obtain a predicted structure of other BoNT serotypes based on the BoNT/A1 crystal structure (3BTA.pdb);
2. The structural (pdb) files thus obtained may be edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) may be used to superpose each serotype onto the 3BTA.pdb structure;
4. The superposed pdb files may be inspected to locate the 3₁₀ helix at the start of the H_{C} domain of BoNT/A1, and corresponding residues in the other serotype may then identified.
5. The other BoNT serotype sequences may be aligned with Clustal Omega in order to check that corresponding residues were correct.

Examples of LH_{N}, H_{C} and 3₁₀ helix domains determined by this method are presented below:

| | Accession | | | |
|---|---|---|---|---|
| | Number | | | |
| | (Plus Sequence | | | |
| | Version after | | | |
| Neurotoxin | Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | |
| NO: 2) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (SEQ ID | | | | |
| NO: 8) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A212S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | 16Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8th residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a H_{C} domain from BoNT/B, wherein the C-terminal amino acid residue of the LH_{N} domain corresponds to the eighth amino acid residue N-terminally to the β-strand located at the beginning (N-term) of the H_{C} domain of BoNT/A, and wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a H_{C} domain from BoNT/B, wherein the C-terminal amino acid residue of the LH_{N} domain corresponds to the C-terminal amino acid residue of the α-helix located at the end (C-terminus) of the LH_{N} domain of BoNT/A, and wherein the N-terminal amino acid residue of the H_{C} domain corresponds to the amino acid residue immediately C-terminal to the C-terminal amino acid residue of the α-helix located at the end (C-terminus) of the LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the modified BoNT/A (e.g. chimeric neurotoxin), thereby allowing for the modified BoNT/A (e.g. chimeric neurotoxin) to exert its functions to their full capacity.

In fact, surprisingly, retaining solely the first amino acid residue of the 3₁₀ helix of the BoNT/A and the second amino acid residue of the 3₁₀ helix onwards of BoNT/B not only allows the production of soluble and functional BoNT/AB chimera, but further leads to improved properties over other BoNT/AB chimeras, in particular an increased potency, an increased Safety Ratio and/or a longer duration of action (as well as increased Safety Ratio and/or duration of action when compared to unmodified BoNT/A—e.g. SEQ ID NO: 2, such as SEQ ID NO: 2 in a di-chain form).

The BoNT/A light-chain, BoNT/A translocation domain, and/or BoNT/B H_{C} domain may be a modified BoNT/A light-chain, BoNT/A translocation domain, and/or BoNT/B H_{C} domain or a derivative thereof, including but not limited to those described below. A modified BoNT/A light-chain, BoNT/A translocation domain, and/or BoNT/B H_{C} domain or derivative may contain one or more amino acids that has been modified as compared to the native (unmodified) form of the BoNT/A light-chain, BoNT/A translocation domain, and/or BoNT/B H_{C} domain, or may contain one or more inserted amino acids that are not present in the native (unmodified) form of the BoNT/A light-chain, BoNT/A translocation domain, and/or BoNT/B H_{C} domain. By way of example, a modified BoNT/A light-chain, BoNT/A translocation domain, and/or BoNT/B H_{C} domain may have modified amino acid sequences in one or more domains relative to the native (unmodified) BoNT/A light-chain, BoNT/A translocation domain, and/or BoNT/B H_{C} domain sequence. Such modifications may modify functional aspects thereof, for example biological activity or persistence. Thus, in one embodiment, the BoNT/A light-chain, BoNT/A translocation domain, and/or BoNT/B H_{C} domain is a modified BoNT/A light-chain, BoNT/A translocation domain, and/or BoNT/B H_{C} domain, or modified BoNT/A light-chain, BoNT/A translocation domain, and/or BoNT/B H_{C} domain derivative.

A modified BoNT/B H_{C} domain may have one or more modifications modifying binding to target nerve cells, for example providing higher or lower affinity binding when compared to the native (unmodified) BoNT/B H_{C} domain. Such modifications in the BoNT/B H_{C} domain may include modifying residues in the ganglioside binding site of the H_{C} domain or in the protein (e.g. synaptotagmin) binding site that alter binding to the ganglioside receptor and/or the protein receptor of the target nerve cell. Examples of such modified neurotoxins are described in WO 2006/027207 and WO 2006/114308, both of which are hereby incorporated by reference in their entirety.

A modified light-chain may have one or more modifications in the amino acid sequence thereof, for example modifications in the substrate binding or catalytic domain which may alter or modify the SNARE protein specificity of the modified light-chain, preferably with the proviso that said modifications do not catalytically inactivate said light-chain. Examples of such modified neurotoxins are described in WO 2010/120766 and US 2011/0318385, both of which are hereby incorporated by reference in their entirety.

The LH_{N} domain from BoNT/A may correspond to amino acid residues 1 to 872 of SEQ ID NO: 2, or a polypeptide sequence having at least 70% sequence identity thereto. The LH_{N} domain from BoNT/A may correspond to amino acid residues 1 to 872 of SEQ ID NO: 2, or a polypeptide sequence having at least 80%, 90% or 95% sequence identity thereto. Preferably, the LH_{N} domain from BoNT/A corresponds to amino acid residues 1 to 872 of SEQ ID NO: 2.

The H_{C} domain from BoNT/B may correspond to amino acid residues 860 to 1291 of SEQ ID NO: 8, or a polypeptide sequence having at least 70% sequence identity thereto. The H_{C} domain from BoNT/B may correspond to amino acid residues 860 to 1291 of SEQ ID NO: 8, or a polypeptide sequence having at least 80%, 90% or 95% sequence identity thereto. Preferably, the H_{C} domain from BoNT/B corresponds to amino acid residues 860 to 1291 of SEQ ID NO: 8.

Preferably, the BoNT/AB chimera comprises a BoNT/A1 LH_{N} domain and a BoNT/B1 H_{C} domain. More preferably, the LH_{N} domain corresponds to amino acid residues 1 to 872 of BoNT/A1 (SEQ ID NO: 2) and the H_{C} domain corresponds to amino acid residues 860 to 1291 of BoNT/B1 (SEQ ID NO: 8).

Most preferably, a BoNT/B H_{C} domain further comprises at least one amino acid residue substitution, insertion, indel or deletion in the H_{CC} subdomain which has the effect of increasing the binding affinity of BoNT/B neurotoxin for human Syt II as compared to the natural BoNT/B sequence. Suitable amino acid residue substitutions, insertions, indels or deletions in the BoNT/B H_{CC} subdomain have been disclosed in WO 2013/180799 and in WO 2016/154534 (both herein incorporated by reference).

A suitable amino acid residue substitution, insertion, indel or deletion in the BoNT/B H_{CC} subdomain may include a substitution mutation selected from the group consisting of: V1118M; Y1183M; E1191M; E1191I; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

A suitable amino acid residue substitution, insertion, indel or deletion in the BoNT/B H_{CC} subdomain may further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

A suitable amino acid residue substitution, insertion, indel or deletion in the BoNT/B H_{CC} subdomain may also include a combination of three substitution mutations which are E1191M, S1199W and W1178Q.

Preferably, the amino acid residue substitution, insertion, indel or deletion in the BoNT/B H_{CC} subdomain includes a combination of two substitution mutations which are E1191M and S1199Y. Such modifications are present in modified BoNT/A (e.g. BoNT/AB chimeras) of SEQ ID NO: 5 and SEQ ID NO: 6, for example. E1191M may correspond to position 1204 of SEQ ID NO: 6 and S1199Y may correspond to position 1212. Thus, SEQ ID NO: 6 may comprise 1204M and 1212Y.

The modification may be a modification when compared to unmodified BoNT/B shown as SEQ ID NO: 8, wherein the amino acid residue numbering is determined by alignment with SEQ ID NO: 8. As the presence of a methionine residue at position 1 of SEQ ID NO: 8 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 8 includes a methionine, the position numbering will be as defined above (e.g. E1191 will be E1191 of SEQ ID NO: 8). Alternatively, where the methionine is absent from SEQ ID NO: 8 the amino acid residue numbering should be modified by −1 (e.g. E1191 will be E1190 of SEQ ID NO: 8). Accordingly, an initial methionine amino acid residue of a polypeptide sequence of the modified BoNT/A may be optional or absent. Similar considerations apply when the methionine at position 1 of the other polypeptide sequences described herein is present/absent, and the skilled person will readily determine the correct amino acid residue numbering using techniques routine in the art.

A modified BoNT/A for use in the invention may comprise a polypeptide sequence having at least 70% sequence identity to a polypeptide sequence selected from SEQ ID NOs: 3-7. For example, a polypeptide sequence having at least 80%, 90%, 95% or 99.9% sequence identity to a polypeptide sequence selected from SEQ ID NOs: 3-7. Preferably, a modified BoNT/A for use in the invention may comprise (more preferably consist of) a polypeptide sequence selected from SEQ ID NOs: 3-7.

It is preferred that the modified BoNT/A comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/A for use in the invention may comprise (more preferably consist of) SEQ ID NO: 6.

The term “deletion” as used herein refers to removal of one or more amino acid residues of a polypeptide without replacement of one or more amino acid residues at the site of deletion. Thus, where one amino acid residue has been deleted from a polypeptide sequence having x number of amino acid residues (for example), the resultant polypeptide has x−1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x−1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues.

Methods for modifying proteins by substitution, insertion or deletion of amino acid residues are known in the art. By way of example, amino acid modifications may be introduced by modification of a DNA sequence encoding a BoNT/A (e.g. encoding unmodified BoNT/A). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised. Typically a modification may be carried out by either modifying a nucleic acid encoding a native clostridial neurotoxin (or part thereof) such that the modified BoNT/A (or part thereof) encoded by the nucleic acid comprises the modification(s). Alternatively, a nucleic acid that encodes a modified clostridial neurotoxin (or part thereof) comprising the modification(s) may be synthesized.

Where a polypeptide sequence of a modified BoNT/A described herein comprises a tag, e.g. for purification, such as a His-tag, said tag is optional. Preferably, said tag is removed prior to use of the modified BoNT/A according to the invention.

As discussed above, a modified BoNT/A described herein has increased tissue retention properties that also provide increased potency and/or duration of action and can allow for increased dosages without any additional negative effects. One way in which these advantageous properties may be defined is in terms of the Safety Ratio of the modified BoNT/A. In this regard, undesired effects of a clostridial toxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial toxin can be assessed experimentally by Digital Abduction Score (DAS) assay, a measurement of muscle paralysis. The DAS assay may be performed by injection of 20 μl of clostridial neurotoxin, formulated in Gelatin Phosphate Buffer, into the mouse gastrocnemius/soleus complex, followed by assessment of Digital Abduction Score using the method of Aoki (Aoki K R, Toxicon 39:1815-1820; 2001). In the DAS assay, mice are suspended briefly by the tail in order to elicit a characteristic startle response in which the mouse extends its hind limbs and abducts its hind digits. Following clostridial neurotoxin injection, the varying degrees of digit abduction are scored on a five-point scale (0=normal to 4=maximal reduction in digit abduction and leg extension).

The Safety Ratio of a neurotoxin (e.g. modified BoNT/A of the invention (or unmodified BoNT/A for comparison)) may then be expressed as the ratio between the amount of toxin required for a 10% drop in a bodyweight (measured at peak effect within the first seven days after dosing in a mouse) and the amount of neurotoxin required for a DAS score of 2. High Safety Ratio scores are therefore desired and indicate a neurotoxin that is able to effectively paralyze a target muscle with little undesired off-target effects. A modified BoNT/A of the present invention has a Safety Ratio that is higher than the Safety Ratio of an equivalent unmodified (native) BoNT/A.

A high Safety Ratio is particularly advantageous in therapy because it represents an increase in the therapeutic index. In other words, this means that reduced dosages can be used compared to alternative clostridial neurotoxin therapeutics and/or that increased dosages can be used without any additional (e.g. deleterious) effects. The possibility to use higher doses of neurotoxin without additional effects is particularly advantageous as higher doses usually lead to a longer duration of action of the neurotoxin.

The potency of a modified BoNT/A may be expressed as the minimal dose of neurotoxin which leads to a given DAS score when administered to a mouse gastrocnemius/soleus complex, for example a DAS score of 2 (ED₅₀ dose) or a DAS score of 4. The Potency of a modified BoNT/A may be also expressed as the EC₅₀ dose in a cellular assay measuring SNARE cleavage by the neurotoxin, for example the EC₅₀ dose in a cellular assay measuring SNAP25 cleavage by a modified BoNT/A.

The duration of action of a modified BoNT/A may be expressed as the time required for retrieving a DAS score of 0 after administration of a given dose of neurotoxin, for example the minimal dose of neurotoxin leading to a DAS score of 4, to a mouse gastrocnemius/soleus complex.

Thus, in one embodiment, a modified BoNT/A of the present invention has a Safety Ratio that is greater than 7 (for example, at least 8, 9, 10, 15, 20, 25, 30, 35, 40, 45 or 50), wherein Safety Ratio is calculated as: dose of toxin required for-10% bodyweight change (pg/mouse) divided by DAS ED₅₀ (pg/mouse) [ED₅₀=dose required to produce a DAS score of 2]. For example, a modified BoNT/A may have a Safety Ratio of at least 8, 9, 10, 15, 20, 25, 30, 35, 40, 45 or 50.

In one embodiment, a modified BoNT/A of the present invention has a Safety Ratio of at least 10. In one embodiment, a modified BoNT/A of the present invention has a Safety Ratio of at least 15.

Preferably, the modified BoNT/A has a Safety Ratio of at least 10 (e.g. a Safety Ratio of 10), more preferably at least 12 or 13 (e.g. 14-15).

The modified BoNT/A may have a Safety Ratio of greater than 7 up to 50 e.g. 8-45, 10-20 or 12-15.

In use, the modified BoNT/A of the invention is in a di-chain form.

A modified BoNT/A for use in the invention may comprise a polypeptide sequence having at least 70% sequence identity to a polypeptide sequence selected from SEQ ID NOs: 3-7. For example, a polypeptide sequence having at least 80%, 90%, 95% or 99.9% sequence identity to a polypeptide sequence selected from SEQ ID NOs: 3-7. Preferably, a modified BoNT/A for use in the invention may comprise (more preferably consist of) a polypeptide sequence selected from SEQ ID NOs: 3-7. Of said modified BoNT/A neurotoxins (e.g. chimeric clostridial neurotoxins), SEQ ID NO: 6 is preferred.

Thus, it is preferred that the modified BoNT/A (e.g. chimeric clostridial neurotoxin) comprises a polypeptide sequence having at least 70% sequence identity to SEQ ID NO: 6. More preferably, a polypeptide sequence having at least 80%, 90%, 95% or 99.9% sequence identity to SEQ ID NO: 6. Most preferably, a modified BoNT/A (e.g. chimeric clostridial neurotoxin) for use in the invention may comprise (more preferably consist of) SEQ ID NO: 6.

A di-chain modified BoNT/A of the invention may comprise an L-chain portion of a polypeptide sequence having at least 70%, 80%, 90%, 95%, 99.9%, or 100% sequence identity to any one of SEQ ID NOs: 3-7 constituting a first chain of the di-chain modified BoNT/A, and may comprise the H_{N} and H_{C} domains of a polypeptide sequence having at least 70%, 80%, 90%, 95%, 99.9%, or 100% sequence identity to any one of SEQ ID NOS: 3-7 together constituting a second chain of the di-chain modified BoNT/A, wherein the first and second chains are joined together by a di-sulphide bond.

Where cleavage occurs at more than one position (preferably at two positions) within the activation loop of a modified BoNT/A comprising a polypeptide sequence having at least 70%, 80%, 90%, 95%, 99.9%, or 100% sequence identity to any one of SEQ ID NOs: 3-7, a small fragment of the C-terminal L-chain portion of the sequence having at least 70%, 80%, 90%, 95%, 99.9%, or 100% sequence identity to any one of SEQ ID NOs: 3-7 may be absent from the di-chain modified BoNT/A. In view of this, the sequence of the di-chain modified BoNT/A (e.g. comprising a polypeptide sequence having at least 70%, 80%, 90%, 95%, 99.9%, or 100% sequence identity to any one of SEQ ID NOs: 3-7) may be slightly different to that of the corresponding single-chain modified BoNT/A comprising a polypeptide sequence having at least 70%, 80%, 90%, 95%, 99.9%, or 100% sequence identity to any one of SEQ ID NOs: 3-7. The small fragment may be 1-15 amino acids. In particular, in one embodiment, when Lys-C is used to covert a single-chain modified BoNT/A into a di-chain clostridial neurotoxin, the small fragment of the C-terminal L-chain portion of the sequence that is absent may be SEQ ID NO: 9 or 10.

Preferably, a di-chain modified BoNT/A of the invention may comprise an L-chain portion of a polypeptide sequence having at least 70%, 80%, 90%, 95%, 99.9%, or 100% sequence identity to SEQ ID NO: 6 constituting a first chain of the di-chain modified BoNT/A, and may comprise the H_{N} and H_{C} domains of a polypeptide sequence having at least 70%, 80%, 90%, 95%, 99.9%, or 100% sequence identity to SEQ ID NO: 6 together constituting a second chain of the di-chain modified BoNT/A, wherein the first and second chains are joined together by a di-sulphide bond.

Where cleavage occurs at more than one position (preferably at two positions) within the activation loop of a modified BoNT/A comprising a polypeptide sequence having at least 70%, 80%, 90%, 95%, 99.9%, or 100% sequence identity to SEQ ID NO: 6, a small fragment of the C-terminal L-chain portion of the sequence having at least 70%, 80%, 90%, 95%, 99.9%, or 100% sequence identity to SEQ ID NO: 6 may be absent from the di-chain modified BoNT/A. In view of this, the sequence of the di-chain modified BoNT/A (e.g. comprising a polypeptide sequence having at least 70%, 80%, 90%, 95%, 99.9%, or 100% sequence identity to SEQ ID NO: 6) may be slightly different to that of the corresponding single-chain modified BoNT/A comprising a polypeptide sequence having at least 70%, 80%, 90%, 95%, 99.9%, or 100% sequence identity to SEQ ID NO: 6. The small fragment may be 1-15 amino acids. In particular, in one embodiment, when Lys-C is used to covert a single-chain modified BoNT/A into a di-chain modified BoNT/A, the small fragment of the C-terminal L-chain portion of the sequence that is absent may be SEQ ID NO: 9 or 10.

In a particularly preferred embodiment, a di-chain modified BoNT/A comprises (or consists of) a light-chain comprising a polypeptide sequence having at least 70%, 80%, 90%, 95%, or 99.9% sequence identity to SEQ ID NO: 11 or 12 (preferably SEQ ID NO: 11) and a heavy-chain comprising a polypeptide sequence having at least 70%, 80%, 90%, 95%, or 99.9% sequence identity to SEQ ID NO: 13, wherein the light-chain and heavy-chain are joined together by a di-sulphide bond. More preferably, a di-chain modified BoNT/A comprises (or consists of) a light-chain comprising SEQ ID NO: 11 or 12 (preferably SEQ ID NO: 11) and a heavy-chain comprising SEQ ID NO: 13, wherein the light-chain and heavy-chain are joined together by a di-sulphide bond. Even more preferably, a di-chain modified BoNT/A comprises (or consists of) a light-chain having SEQ ID NO: 11 and a heavy-chain having SEQ ID NO: 13, wherein the light-chain and heavy-chain are joined together by a di-sulphide bond. The di-sulphide bond is preferably formed by and/or is between cysteine residue 429 of SEQ ID NO: 11 or 12 and cysteine residue 6 of SEQ ID NO: 13.

In a preferred embodiment, a modified BoNT/A (e.g. chimeric clostridial neurotoxin) of the invention does not comprise a therapeutic or diagnostic agent (e.g. a nucleic acid, protein, peptide or small molecule therapeutic or diagnostic agent) additional to the light-chain and heavy-chain. For example, in one embodiment, the modified BoNT/A (e.g. chimeric clostridial neurotoxin) may not comprise a covalently or non-covalently associated therapeutic or diagnostic agent. Thus, a modified BoNT/A (e.g. chimeric clostridial neurotoxin) of the invention preferably does not function as a delivery vehicle for a further therapeutic or diagnostic agent.

In embodiments where a modified BoNT/A (e.g. chimeric clostridial neurotoxin) described herein has a tag for purification (e.g. a His-tag) and/or a linker, said tag and/or linker are optional.

The modified BoNT/A is preferably in a non-complexed form (i.e. may be free from complexing proteins that are present in naturally occurring clostridial neurotoxin complex e.g. BoNT/A complex). Examples of such complexing proteins include a neurotoxin-associated proteins (NAP) and a nontoxic-nonhemagglutinin component (NTNH). However, it is preferred that the modified BoNT/A is a recombinant modified BoNT/A. The modified BoNT/A of the present invention can be produced using recombinant nucleic acid technologies. Thus, in one embodiment, a modified BoNT/A (as described herein) is a recombinant modified BoNT/A (e.g. chimeric clostridial neurotoxin).

In one embodiment a nucleic acid (for example, DNA) comprising a nucleic acid sequence encoding a modified BoNT/A is provided. In one embodiment, the nucleic acid sequence is prepared as part of a DNA vector comprising a promoter and a terminator. The nucleic acid sequence may be selected from any of the nucleic acid sequences described herein.

In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

In another preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

The nucleic acid molecules may be made using any suitable process known in the art. Thus, the nucleic acid molecules may be made using chemical synthesis techniques. Alternatively, the nucleic acid molecules of the invention may be made using molecular biology techniques.

The DNA construct of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

A modified BoNT/A of the invention may be present as a single-chain or as a di-chain. However, it is preferred that the modified BoNT/A is present as a di-chain in which the L-chain is linked to the H-chain (or component thereof, e.g. the H_{N} domain) via a di-sulphide bond.

Production of a single-chain modified BoNT/A having a light-chain and a heavy-chain may be achieved using a method comprising expressing a nucleic acid encoding a modified BoNT/A in an expression host, lysing the host cell to provide a host cell homogenate containing the single-chain modified BoNT/A, and isolating the single-chain modified BoNT/A. The single-chain modified BoNT/A described herein may be proteolytically processed using a method comprising contacting a single-chain modified BoNT/A with a protease (e.g. Lys-C) that hydrolyses a peptide bond in the activation loop of the modified BoNT/A, thereby converting the single-chain modified BoNT/A into a corresponding di-chain modified BoNT/A (e.g. wherein the light-chain and heavy-chain are joined together by a disulphide bond). A di-chain modified BoNT/A is preferably obtainable by such a method.

The term “obtainable” as used herein also encompasses the term “obtained”. In one embodiment the term “obtainable” means obtained.

Thus, a modified BoNT/A used in the invention is preferably a di-chain modified BoNT/A that has been produced from a single-chain BoNT/A, wherein the single-chain BoNT/A comprises or consists of a polypeptide sequence described herein. For example, it is preferred that the modified BoNT/A used in the invention is a di-chain modified BoNT/A that has been produced from a polypeptide comprising a polypeptide sequence having at least 70% (e.g. at least 80%, 90%, 95% or 99.9%) sequence identity to SEQ ID NO: 6. Most preferably, the modified BoNT/A used in the invention is a di-chain modified BoNT/A that has been produced from a polypeptide comprising (even more preferably consisting of) SEQ ID NO: 6. Accordingly, in some embodiments, the modified BoNT/A is a di-chain modified BoNT/A in which the light-chain (L-chain) is linked to the heavy-chain (H-chain) via a di-sulphide bond obtainable by a method comprising contacting a single-chain modified BoNT/A with a protease that hydrolyses a peptide bond in the activation loop thereof, thereby converting the single-chain modified BoNT/A into the corresponding di-chain modified BoNT/A. Accordingly, in some embodiments, the modified BoNT/A is a di-chain modified BoNT/A in which the light-chain (L-chain) is linked to the heavy-chain (H-chain) via a di-sulphide bond obtainable by a method comprising contacting a single-chain modified BoNT/A comprising SEQ ID NO: 6 with a protease that hydrolyses a peptide bond in the activation loop thereof, thereby converting the single-chain modified BoNT/A into the corresponding di-chain modified BoNT/A. In some embodiments, the modified BoNT/A is a di-chain modified BoNT/A in which the L-chain is linked to the H-chain via a di-sulphide bond obtainable by a method comprising contacting a single-chain modified BoNT/A consisting of SEQ ID NO: 6 with a protease that hydrolyses a peptide bond in the activation loop thereof, thereby converting the single-chain modified BoNT/A into the corresponding di-chain modified BoNT/A.

The protease used to cleave the activation loop is preferably Lys-C. Suitable proteases and methods for cleaving activation loops to produce di-chain clostridial neurotoxins are taught in WO 2014/080206, WO2014/079495, and EP2677029A2, which are incorporated herein by reference. Lys-C may cleave an activation loop C-terminal to one or more of the lysine residues present therein. Where Lys-C cleaves the activation loop more than once, the skilled person will appreciate that a small peptide of the activation loop of a di-chain modified BoNT/A may be absent when compared to a SEQ ID NO shown herein.

The term “one or more” as used herein may mean at least 2, 3, 4, 5, 6, 7, 8, 9, 10, 15, or 20. In one embodiment, wherein “one or more” precedes a list, “one or more” may mean all of the members of the list. Similarly, the term “at least one” as used herein may mean at least 2, 3, 4, 5, 6, 7, 8, 9, 10, 15, or 20. In one embodiment, wherein “at least one” precedes a list, “at least one” may mean all of the members of the list.

The term “disorder” as used herein also encompasses a “disease”. In one embodiment the disorder is a disease.

The modified BoNT/A of the invention may be formulated in any suitable manner for administration to a subject, for example as part of a pharmaceutical composition. Such a pharmaceutical composition comprising a modified BoNT/A of the invention and a pharmaceutically acceptable carrier, excipient, adjuvant, propellant and/or salt.

Fluid dosage forms are typically prepared utilising the modified BoNT/A and a pyrogen-free sterile vehicle. The modified BoNT/A, depending on the vehicle and concentration used, can be either dissolved or suspended in the vehicle. In preparing solutions the modified BoNT/A can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone may be included in the composition(s) to facilitate uniform distribution of the components.

In one embodiment, the invention provides a unit dosage form of modified botulinum neurotoxin A (BoNT/A) for treating upper facial lines, comprising:
a. greater than 73 Units of modified BoNT/A; or
b. greater than 1754 pg (preferably greater than 1800 pg) of modified BoNT/A; and
c. optionally a pharmaceutically acceptable carrier, excipient, adjuvant, and/or salt, wherein the modified BoNT/A comprises a BoNT/A light-chain and translocation domain, and a BoNT/B receptor binding domain (H_{C} domain).

In one aspect, the invention provides a unit dosage form of modified botulinum neurotoxin A (BoNT/A) for treating upper facial lines, the unit dosage form comprising:
a. greater than 73 Units to 332.8 Units of modified BoNT/A, wherein 1 Unit is an amount of the modified BoNT/A that corresponds to the calculated median lethal dose (LD₅₀) in mice; or
b. greater than 1754 pg (preferably greater than 1800 pg) to 8000 pg of modified BoNT/A; and
c. optionally a pharmaceutically acceptable carrier, excipient, adjuvant, and/or salt,
d. wherein the modified BoNT/A comprises a BoNT/A light-chain and translocation domain (H_{N} domain), and a BoNT/B receptor binding domain (H_{C} domain).

Said unit dosage form may comprise:
a. 100 Units to 200 Units of modified BoNT/A; or
b. 4000 pg to 6000 pg of modified BoNT/A.

A unit dosage form for treating facial lines may comprise 73 Units to 332.8 Units of modified BoNT/A. An upper limit said range may be 300, 250, 200, 150 or 100 Units of modified BoNT/A, preferably the upper limit is 200 Units. A lower limit of said range may be 80, 90, 100, 150, 200 or 250 Units of modified BoNT/A, preferably the lower limit is 100 Units. Preferably, a unit dosage form comprises 90 to 250 Units of modified BoNT/A, for example 100 Units to 200 Units.

A unit dosage form for treating facial lines may comprise 1755 pg to 8000 pg of modified BoNT/A. An upper limit of said range may be 7500, 7000, 6500, 6000, 5500, 5000, 4500, 4000, 3500 or 3000 pg of modified BoNT/A, preferably the upper limit is 7000 pg. A lower limit of said range may be 1800, 2000, 2,500, 3,000, 3,500, 4,000, 4,500 or 5,000 pg of modified BoNT/A, preferably the lower limit is 2000 pg. Preferably, a unit dosage form comprises 2000 pg to 7000 pg of modified BoNT/A, e.g. 4,000 pg to 6,000 pg.

A unit dosage form may comprise 1800-7000 pg, 2000-6500 pg, 2500-6000 of modified BoNT/A, preferably 4000-5500 pg of modified BoNT/A.

A unit dosage from may comprise greater than 1754 pg to 8000 pg of modified BoNT/A. An upper limit of the unit dose form may be 8,000, 7,000, 6,000, 5,000, 4,000, 3,000, 2,000 or 1,000 pg of modified BoNT/A, preferably the upper limit is 5,000 pg. A lower limit of the unit dose form may be 1,800, 2,000, 2,500, 3000, or 4,000 pg of modified BoNT/A, preferably the lower limit is 1,800 pg of modified BoNT/A. The unit dose form may comprise: 1,800 to 8,000 pg, or 2,000 to 6,000 pg of modified BoNT/A, more preferably 3,000 to 6,000 pg of modified BoNT/A. The unit dose form may be 4,500 to 5,500 pg of modified BoNT/A, e.g. 4,900 to 5,100 pg of modified BoNT/A.

The unit dosage form may comprise greater than 1800 pg of modified BoNT/A. The unit dosage form may comprise greater than 1800 pg up to 7000 pg of modified BoNT/A, e.g. greater than 1800 pg up to 6000 pg of modified BoNT/A. The unit dosage form may comprise greater than 2000 pg up to 5000 pg of modified BoNT/A.

The unit dosage form may comprise greater than 2555 pg. For example, the unit dosage form may comprise greater than 2555 pg up to 8000 pg of modified BoNT/A, for example greater than 2555 pg up to 6000 pg of modified BoNT/A.

The unit dosage form may comprise 2000 pg, 2500 pg, 3000 pg, 4000 pg, 4500 pg, 5000 pg, 5500 pg, 6000 pg, 6500 pg, 7000 pg, 7500 pg or 8000 pg of modified BoNT/A.

It is more preferred that the unit dosage form may comprise 2000 to 3500 pg of modified BoNT/A. For example, the unit dosage form may comprise 2250 pg to 3250 pg of modified BoNT/A. A particularly preferred unit dosage form may comprise about 2500 pg (e.g. 2500 pg±10%) of modified BoNT/A, for example the unit dosage form may preferably comprise 2500 pg of modified BoNT/A. Another suitable unit dosage form may comprise about 3000 pg (e.g. 3000 pg±10%) of modified BoNT/A, for example the unit dosage form may comprise 3000 pg of modified BoNT/A.

The unit dosage form may comprise 3500-5500 pg of modified BoNT/A. For example, the unit dosage form may comprise 3800 pg to 4200 pg of modified BoNT/A. The unit dosage form may comprise 4800 to 5200 pg of modified BoNT/A. A particularly suitable unit dosage form may comprise about 4000 pg (e.g. 4000 pg±10%) of modified BoNT/A, for example the unit dosage form may comprise 4000 pg of modified BoNT/A. Another particularly preferred unit dosage form may comprise about 5000 pg (e.g. 5000 pg±10%) of modified BoNT/A, for example the unit dosage form may preferably comprise 5000 pg of modified BoNT/A.

Also provided a kit comprising:
a. the unit dosage form as hereinbefore defined; and
b. instructions for use of the same in treating facial lines; and
c. optionally a diluent.

Also provided a kit comprising:
a. the unit dosage form as hereinbefore defined; and
b. instructions for use of the same in treating glabellar lines; and
c. optionally a diluent.

Also provided a kit comprising:
a. the unit dosage form as hereinbefore defined; and
b. instructions for use of the same in treating forehead lines; and
c. optionally a diluent.

Also provided a kit comprising:
a. the unit dosage form as hereinbefore defined; and
b. instructions for use of the same in treating lateral canthal lines; and
c. optionally a diluent.

Also provided a kit comprising:
a. the unit dosage form as hereinbefore defined; and
b. instructions for use of the same in treating glabellar lines, forehead lines and lateral canthal lines; and
c. optionally a diluent.

The modified BoNT/A of the unit dosage form may comprise a polypeptide sequence having at least 70% sequence identity to any one of SEQ ID NOs: 3-7. For example, a polypeptide sequence having at least 80%, 90%, 95% or 99.9% sequence identity to any one of SEQ ID NOs: 3-7.

It is preferred that the modified BoNT/A of the unit dosage form comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/may comprise (more preferably consist of) SEQ ID NO: 6.

Embodiments related to the various therapeutic uses of the invention can be applied to the methods, compositions (e.g. unit dosage forms), and kits of the invention and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M-A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a botulinum neurotoxin A” includes a plurality of such candidate agents and reference to “the botulinum neurotoxin A” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows the FDA approved dosages of Dysport® for treating facial lines in adults.

FIG. 2 shows SDS-PAGE of purified recombinant BoNT/AB chimera 1, 2 and 3A (SEQ ID NO: 3-4, respectively). Lanes are labelled “Marker” (molecular weight marker), “−DTT” (oxidised BoNT/AB chimera sample), and “+DTT” (reduced BoNT/AB chimera sample).

FIG. 3 shows cleavage of SNAP-25 in rat spinal cord neurones by recombinant BoNT/AB chimera 1, 2 and 3A (SEQ ID NO: 3, 4 and 5 converted into a di-chain form, respectively). Cultured rat primary spinal cord neurons (SCN) were exposed to various concentrations of recombinant BoNT/AB chimera 1, 2 or 3A for 24 hours, at 37° C. in a humidified atmosphere with 10% CO₂. Cells were then lysed with 1×NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 4 shows mouse digit abduction scoring assay. Mice were injected into the gastrocnemius-soleus complex muscles of one hind limb, under short general anaesthesia; muscle weakening was measured on a 0-4 scale using the digit abduction score (DAS). DAS max values were determined for each dose and plotted against dose and the data were fitted to a 4-parameter logistic equation, ED50 and dose leading to DAS 4 (DAS 4 dose) values were determined.

FIG. 5 shows SDS-PAGE of purified recombinant BoNT/AB chimera 3B and 3C (SEQ ID NO: 6 and 7 respectively). Lanes are labelled “Marker” (molecular weight marker), “−DTT” (oxidised BoNT/AB chimera sample), and “+DTT” (reduced BoNT/AB chimera sample).

FIG. 6 shows cleavage of SNAP-25 by unmodified BoNT/A and BoNT/AB chimera 3B and 3C (SEQ ID NO: 2, 6 and 7 respectively) in human induced pluripotent stem cell derived peripheral neurons (PERI.4U—Axiogenesis, Germany). PERI.4U cells were exposed to various concentrations of recombinant BoNT/A, or BoNT/AB chimera 3B or 3C for 24 hours, at 37° C. in a humidified CO₂ atmosphere containing 5% CO₂. Cells were then lysed with 1x NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 7 shows duration of muscle weakening over time in the mouse digit abduction scoring assay. Mice were injected into the gastrocnemius-soleus complex muscles of one hind limb, under short general anaesthesia; muscle weakening was measured on a 0-4 scale using the digit abduction score (DAS). Animals of the group injected with the lowest dose that induced during the first four days of injection a DAS of 4 were monitored until complete recovery of the muscle weakness to a DAS of 0 (no observed muscle weakness).

FIG. 8 shows the injection sites for the intramuscular administration of modified BoNT/A when treating glabellar lines.

FIG. 9 shows the injection sites for the intramuscular administration of modified BoNT/A when treating glabellar lines and forehead lines.

FIG. 10 shows the injection sites for the intramuscular administration of modified BoNT/A when treating only lateral canthal lines.

FIG. 11 shows the injection sites for the intramuscular administration of modified BoNT/A when treating glabellar lines, forehead lines and lateral canthal lines.

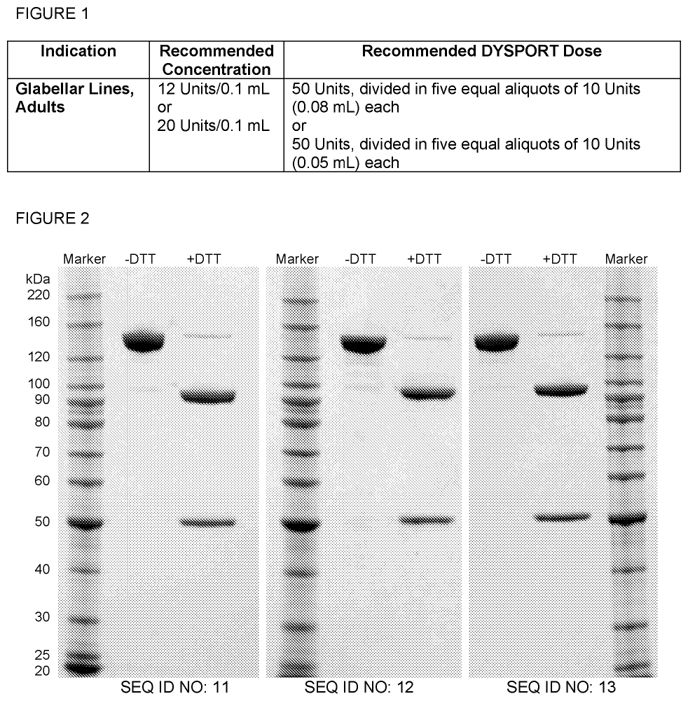

### EXAMPLES

### Example 1

### Cloning, Expression and Purification of BoNT/AB Chimeras

BoNT/AB chimeric constructs 1, 2, 3A, 3B, and 3C (SEQ ID NO: 3-7, respectively) were constructed from DNA encoding the parent serotype molecule and appropriate oligonucleotides using standard molecular biology techniques. These were then cloned into the pJ401 expression vector with or without a C-terminal His₁₀-tag and transformed into BLR (DE3) E. coli cells for over-expression. These cells were grown at 37° C. and 225 RPM shaking in 2 L baffled conical flasks containing 1 L modified Terrific Broth (mTB) supplemented with the appropriate antibiotic. Once the A₆₀₀ reached >0.5, the incubator temperature was decreased to 16° C., and then induced with 1 mM IPTG an hour later for 20 h at 225 RPM shaking, to express the recombinant BoNT/AB construct.

Harvested cells were lysed by ultrasonication and clarified by centrifugation at 4500 RPM for 1 h at 4° C. The recombinant BoNT/AB chimeric molecules were then extracted in ammonium sulphate and purified by standard fast protein liquid chromatography (FPLC) techniques. This involved using a hydrophobic interaction resin for capture and an anion-exchange resin for the intermediate purification step. The partially purified molecules were then proteolytically cleaved with endoproteinase Lys-C to yield the active di-chain. This was further purified with a second hydrophobic interaction resin to obtain the final BoNT/AB chimera.

For BoNT/AB chimeric molecules with a decahistadine tag (H₁₀) (chimera 1, 2, 3A), the capture step employed the use of an immobilised nickel resin instead of the hydrophobic interaction resin.

The sequence of each chimera is presented in Table 1.

TABLE 1
chimeric BoNT/AB constructs

| Molecule | SEQ ID NO | Sequence |
|---|---|---|
| Chimera 1 | 3 | A1:1-871 + B1:858-1291 |
| | | (E1191M/S1199Y) + His₁₀-tag |
| Chimera 2 | 4 | A1:1-874 + ELGGGGSEL + B1:858-1291 |
| | | (E1191M/S1199Y) + His₁₀-tag |
| Chimera 3A | 5 | A1:1-872 + B1: 860-1291 |
| | | (E1191M/S1199Y) + His₁₀-tag |
| Chimera 3B | 6 | A1:1-872 + B1: 860-1291 (E1191M/S1199Y) |
| Chimera 3C | 7 | A1:1-872 + B1: 860-1291 |

### Example 2

### Comparison of BoNT/AB Chimera 1, 2 and 3A

BoNT/AB chimera 1, 2 and 3A which have a C-terminal His₁₀ tag and E1191M/S1199Y double mutation were purified as described in Example 1 (FIG. 2) and tested for functional activity.

### Rat Spinal Cord Neurons SNAP-25 Cleavage Assay

Primary cultures of rat spinal cord neurons (SCN) were prepared and grown, for 3 weeks, in 96 well tissue culture plates (as described in: Masuyer et al., 2011, J. Struct. Biol. Structure and activity of a functional derivative of Clostridium botulinum neurotoxin B; and in: Chaddock et al., 2002, Protein Expr. Purif. Expression and purification of catalytically active, non-toxic endopeptidase derivatives of Clostridium botulinum toxin type A). Serial dilutions of BoNT/AB were prepared in SCN feeding medium. The growth medium from the wells to be treated was collected and filtered (0.2 μm filter). 125 μL of the filtered medium was added back to each test well. 125 μL of diluted toxin was then added to the plate (triplicate wells). The treated cells were incubated at 37° C., 10% CO₂, for 24±1 h).

### Analysis of BoNT Activity Using the SNAP-25 Cleavage Assay

Following treatment, BoNT was removed and cells were washed once in PBS (Gibco, UK). Cells were lysed in 1×NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

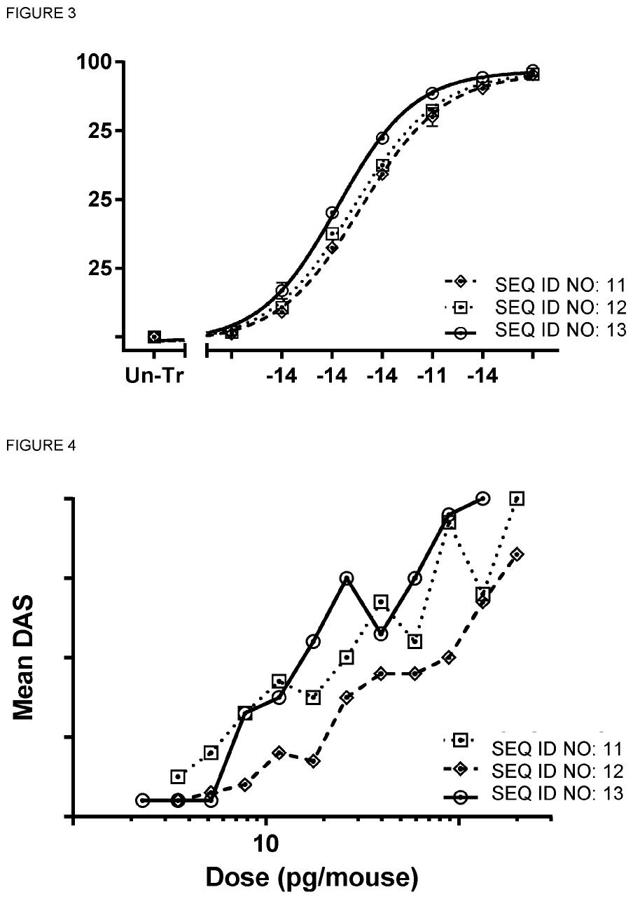

Table 2 below provides the pEC₅₀ values determined for Chimera 1, 2 and 3A in the rat SCN SNAP-25 cleavage assay. These results show that the three BoNT/AB chimeras retained the ability to enter rat spinal cord neurons and cleave their target substrate. However, chimera 3A was more potent than chimera 1 and 2 in this assay (see also FIG. 3).

TABLE 2
pEC₅₀ values.

| | pEC₅₀ ± SEM |
|---|---|
| Chimera 1 | 12.42 ± 0.04 |
| Chimera 2 | 12.57 ± 0.01 |
| Chimera 3A | 12.89 ± 0.04 |

### Digit Abduction Scoring (DAS) Assay

The method to measure the activity of BoNT/AB chimera 1, 2 and 3A in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT/AB chimera or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days.

FIG. 4 shows the fitted curves for chimera 1, 2 and 3A (SEQ ID NO: 3, 4 and 5 converted into a di-chain form, respectively). The chimera 3A curve is shifted to the left, meaning lower doses of chimera 3A achieved a similar DAS response compared to chimera 1 and 2, therefore showing that chimera 3A is more potent than the others in the mouse DAS assay; see also the table below (Table 3) that provides the values for the calculated ED50 and the dose leading to DAS 4 (highest score) for each chimera.

Table 3 below provides the ED₅₀ and DAS 4 doses determined for unmodified recombinant BoNT/A1 (rBoNT/A1—SEQ ID NO: 2 converted into a di-chain form) and chimeras 1, 2 and 3A in the mouse DAS assay. These results show that of the three chimeras, chimera 3A has the highest in vivo potency in inducing muscle weakening. Studies shown in FIG. 4 and Table 3 were performed in mice obtained from Charles River laboratories.

TABLE 3
ED₅₀ values.

| | ED₅₀ | DAS 4 dose |
|---|---|---|
| | (pg/mouse) | (pg/mouse) |
| rBoNT/A 1 | 1 | 5 |
| Chimera 1 | 23 | 200 |
| Chimera 2 | 89 | >300 |
| Chimera 3A | 18 | 133 |

### Example 3

### Comparison of BoNT/AB Chimera 3B, 3C and Unmodified BoNT/A1

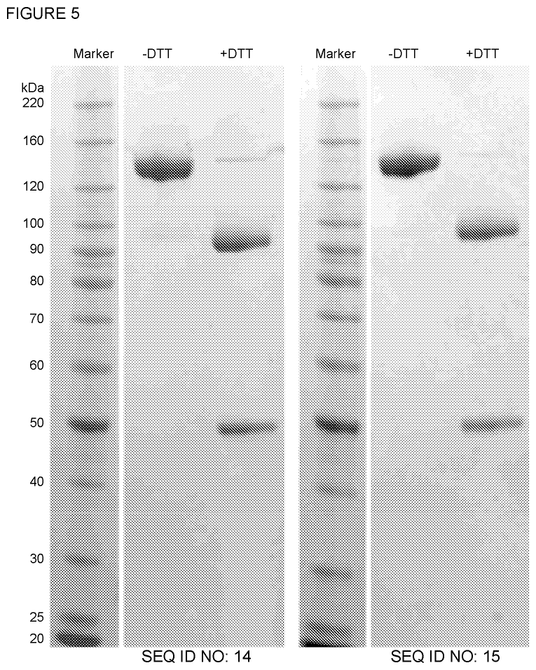

Untagged BoNT/AB chimera 3B and 3C, respectively with and without the presence of the E1191M/S1199Y double mutation (SEQ ID NO: 6 and 7) were purified as described in Example 1 (FIG. 5), and tested for functional activity using unmodified BoNT/A (SEQ ID NO: 2 converted into a di-chain form) as a reference.

### Human Pluripotent Stem Cells Snap-25 Cleavage Assay

Cryopreserved PERI.4U-cells were purchased from Axiogenesis (Cologne, Germany). Thawing and plating of the cells were performed as recommended by the manufacturer.

Briefly, cryovials containing the cells were thawed in a water bath at 37° C. for 2 minutes. After gentle resuspension the cells were transferred to a 50 ml tube. The cryovial was washed with 1 mL of Peri.4U® thawing medium supplied by the manufacturer and the medium was transferred drop-wise to the cell suspension to the 50 ml tube, prior to adding a further 2 mL of Peri.4U® thawing medium drop-wise to the 50 ml tube. Cells were then counted using a hemocytometer. After this, a further 6 mL of Peri.4U® thawing medium was added to the cell suspension. A cell pellet was obtained by centrifugation at 260×g (e.g. 1,100 RPM) for 6 minutes at room temperature. Cells were then resuspended in complete Peri.4U® culture medium supplied by the manufacturer. Cells were plated at a density of 50,000 to 150,000 cells per cm² on cell culture plates coated with poly-L-ornithine and laminin. Cells were cultured at 37° C. in a humidified CO₂ atmosphere, and medium was changed completely every 2-3 days during culture.

For toxin treatment, serial dilutions of BoNTs were prepared in Peri.4U® culture medium. The medium from the wells to be treated was collected and filtered (0.2 μm filter). 125 μL of the filtered medium was added back to each test well. 125 μL of diluted toxin was then added to the plate (triplicate wells). The treated cells were incubated at 37° C., 10% CO₂, for 48±1 h).

### Analysis of BoNT Activity Using the SNAP-25 Cleavage Assay

Following treatment, BoNT was removed and cells were washed once in PBS (Gibco, UK). Cells were lysed in 1×NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

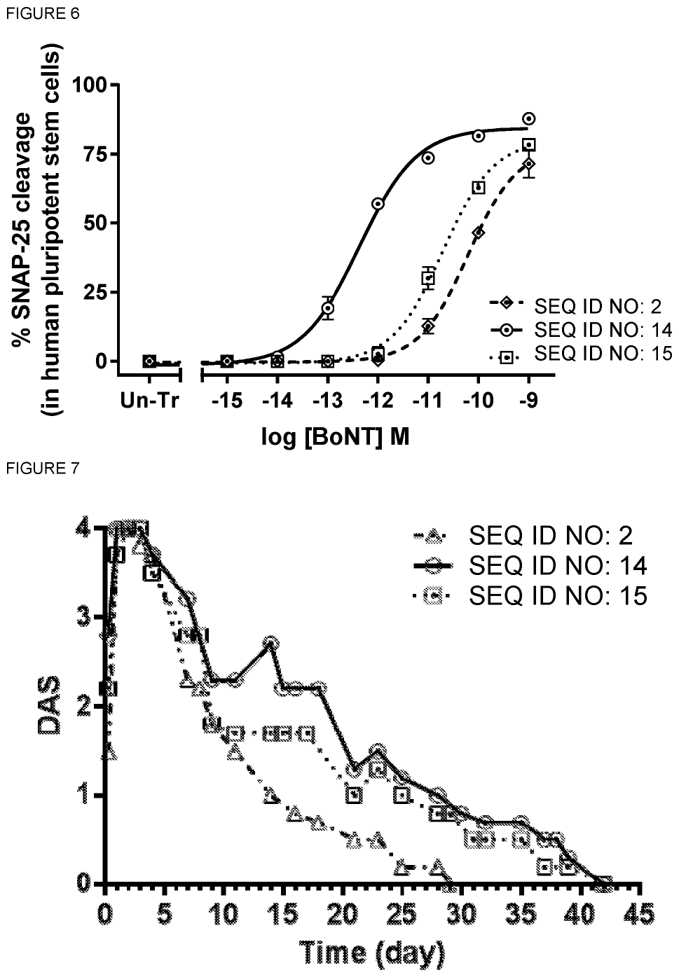

FIG. 6 shows that chimera 3B and 3C displayed greater potency than rBoNT/A1 in cleaving SNAP-25 in induced human pluripotent stem cells but the former significantly more so. This can be explained by the double mutation which increases the affinity of chimera 3B for the human synaptotagmin II protein receptor present in these cells (FIG. 6, Table 4).

TABLE 4
pEC₅₀ values.

| | pEC₅₀ ± SEM |
|---|---|
| rBoNT/A1 | 10.21 ± 0.05 |
| Chimera 3B | 12.38 ± 0.06 |
| Chimera 3C | 10.72 ± 0.08 |

### Digit Abduction Scoring (DAS) Assay—Safety Ratio

The method to measure the activity of BoNTs in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days for all doses. Animals of the groups injected with vehicle and the lowest dose that induced during the first four days of injection a DAS of 4 were thereafter monitored until complete recovery of the muscle weakness to a DAS of 0 (no observed muscle weakness).

For calculation of the safety ratio all animals were weighed the day before toxin injection (D0) and thereafter once daily throughout the duration of the study. The average body weight, its standard deviation, and the standard error mean were calculated daily for each dose-group. To obtain the safety ratio for a BoNT (−10% ΔBW/ED₅₀), the dose at which at any time during the study the average weight of a dose-group was lower than 10% of the average weight at D0 of that same dose-group was divided by the ED₅₀ for the BoNT studied. The lethal dose was defined as the dose at which one or more of the animals within that dose-group died.

FIG. 7 shows the duration of muscle weakening over time in the mouse digit abduction scoring assay for unmodified BoNT/A, chimera 3B and chimera 3C (SEQ ID NO: 2, 6 and 7 converted into a di-chain form), showing that the chimera has longer duration of action.

Table 5 below provides the ED₅₀ and DAS 4 doses determined for rBoNT/A1 and chimeras 3B and 3C in the mouse DAS assay. The table also provide the total duration of action for the DAS 4 dose until complete recovery of the muscle weakness to a DAS of 0 (no observed muscle weakness). In addition, the table shows the mouse lethal dose and the safety ratio (−10% ΔBW/ED₅₀), as defined in the text above. In comparison to rBoNT/A1, chimeras 3B and 3C have longer duration of action, a better safety ratio, and a higher lethal dose. Studies shown in FIG. 7 and Table 5 were performed in mice obtained from Janvier laboratories.

TABLE 5
DAS and Safety Ratios of the BoNT/AB chimeras.

| | | | Total | | |
|---|---|---|---|---|---|
| | ED₅₀ | | duration of | | |
| | (DAS 2) | DAS | action | Mouse | |
| | Dose | 4 dose | (day) with | lethal | Safety ratio |
| | (pg/ | (pg/ | lowest DAS 4 | dose | (−10% |
| | mouse) | mouse) | dose | (pg) | ΔBW/ED₅₀) |
| rBoNT/A1 | 0.9 | 2.3 | 29 | 18 | 4.5 |
| Chimera | 8.0 | 89 | 42 | 200 | 14.1 |
| 3B | | | | | |
| Chimera | 5.0 | 26 | 42 | 8.9 | 7.4 |
| 3C | | | | | |

### Example 4

### Dosage Regimen for Treating Glabellar Lines

Modified BoNT/A is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

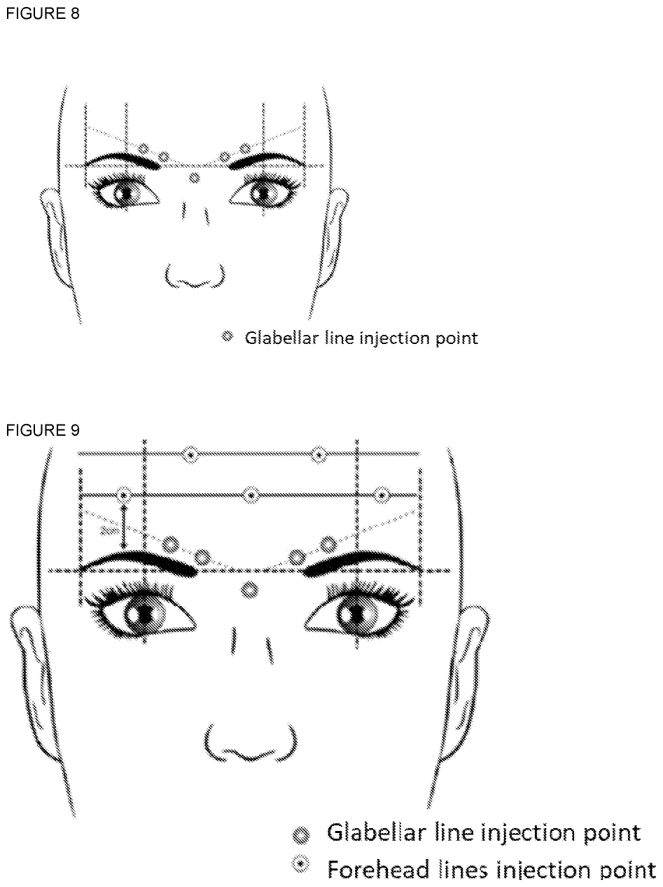

Moderate to severe glabellar lines are treated according to the injection regimen shown in FIG. 8.

The unit dose is 20-300 pg (2-35 Units).

Intramuscular injections are administered at up to five sites according to the unit dose. A maximum total dosage administered is 1500 pg (177 Units).

### Example 5

### Dosage Regimen for Treating Glabellar Lines and Forehead Lines

Modified BoNT/A is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

Moderate to severe glabellar and forehead lines are treated according to the injection regimen shown in FIG. 9.

The unit dose is 20-300 pg (2-35 Units).

Intramuscular injections are administered at up to ten sites according to the unit dose. A maximum total dosage administered is 3000 pg (355 Units).

### Example 6

### Dosage Regimen for Treating Lateral Canthal Lines

Modified BoNT/A is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

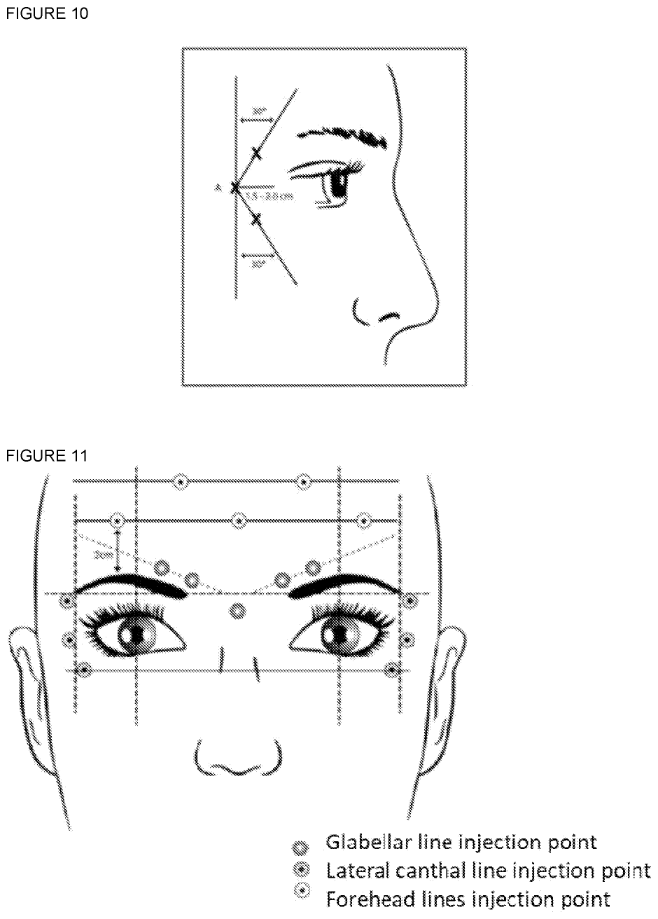

Moderate to severe lateral canthal lines are treated according to the injection regimen shown in FIG. 10.

The unit dose is 20-300 pg (2-35 Units).

Intramuscular injections are administered at up to six sites according to the unit dose. A maximum total dosage administered is 1800 pg (213 Units).

### Example 7

### Dosage Regimen for Treating Glabellar, Forehead and Lateral Canthal Lines

Modified BoNT/A is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

Moderate to severe glabellar, forehead and lateral canthal lines are treated according to the injection regimen shown in FIG. 11.

The unit dose is 20-300 pg (2-35 Units).

Intramuscular injections are administered at up to sixteen sites according to the unit dose. A maximum total dosage administered is 4800 pg (569 Units).

### Example 8

Further Characterisation of a Modified BoNT/A (SEQ ID NO: 6 Converted into a Di-Chain Form)

Modified BoNT/A (e.g. BoNT/AB chimera) SEQ ID NO: 6 converted into a di-chain form was tested in a mouse LD₅₀ assay yielding a result of 1.202 ng/kg. 1 Unit of SEQ ID NO: 6 therefore corresponds to 24.04 pg in this assay.

Additionally, said BoNT/AB chimera was tested in a rat DAS assay to determine the duration of action (as per Example 6) when compared to Dysport®. Results are presented in Table 6 below:

TABLE 6
Duration of action.

| | Dysport ® | BoNT/AB |
|---|---|---|
| | 3 U/rat | 300 pg/rat |
| | 15 U/kg | 1.5 ng/kg |
| Duration of Action (median | 21.9 | 47.7 |
| days) | | |

In conclusion, the duration of action of BoNT/AB was much higher than Dysport® and similar to that of SEQ ID NO: 6. Thus, it is expected that the unit doses and dosage regimen for SEQ ID NO: 6 could similarly be applied to BoNT/AB to provide an improved treatment of facial lines.

### Example 9

Calculation of a Unit Dose of Modified BoNT/A (SEQ ID NO: 6 Converted into a Di-Chain Form) for Upper Facial Lines

In view of pre-clinical pharmacology data, a suitable unit dose range (UD) for administration of modified BoNT/A in humans has been calculated.

A DAS ED₅₀ of 13 pg/kg was calculated for SEQ ID NO: 6. ED₅₀ is considered as a minimal pharmacologically active dose, which is approximately 300-fold lower than the no observed adverse effect level (NOAEL) of 4 ng/kg in the same animal species. An ED₅₀ of 13 pg/kg of SEQ ID NO: 6 in rats corresponds to a 0.8 ng dose for a human of 60 kg body weight.

Thus, the lower limit of a unit dose of 20 pg was selected. An upper limit of the unit dose of 1500 pg was selected, which is lower than the NOAEL of 4 ng/kg from both nonclinical safety species (rat and monkey) converted into human dose for 60 kg body weight.

In view of the improved safety profile, one maximum total dose for the treatment of upper facial lines was set at 24,000 pg, which is derived from the NOAEL of 4 ng/kg from both nonclinical safety species (rat and monkey) converted into human dose for 60 kg body weight.

### Example 10

Dosage Regimen for Treating Glabellar Lines Using a Modified BoNT/A (SEQ ID NO: 6 Converted into a Di-Chain Form)

Modified BoNT/A is provided as a lyophilised powder in a vial containing 36 ng of modified BoNT/A per vial. The lyophilised powder is reconstituted.

Moderate to severe glabellar lines are treated according to the injection regimen shown in FIG. 8.

The unit dose is 20-1500 pg (0.8-62 Units).

Intramuscular injections are administered at up to five sites according to the unit dose. A maximum total dosage administered is 7500 pg (312 Units).

### Example 11

Dosage Regimen for Treating Glabellar Lines and Forehead Lines Using a Modified BoNT/A (SEQ ID NO: 6 Converted into a Di-Chain Form)

Modified BoNT/A is provided as a lyophilised powder in a vial containing 36 ng of modified BoNT/A per vial. The lyophilised powder is reconstituted.

Moderate to severe glabellar and forehead lines are treated according to the injection regimen shown in FIG. 9.

The unit dose is 20-1500 pg (0.8-62 Units).

Intramuscular injections are administered at up to ten sites according to the unit dose. A maximum total dosage administered is 15,000 pg (624 Units).

### Example 12

Dosage Regimen for Treating Lateral Canthal Lines Using a Modified BoNT/A (SEQ ID NO: 6 Converted into a Di-Chain Form)

Modified BoNT/A is provided as a lyophilised powder in a vial containing 36 ng of modified BoNT/A per vial. The lyophilised powder is reconstituted.

Moderate to severe lateral canthal lines are treated according to the injection regimen shown in FIG. 10.

The unit dose is 20-1500 pg (0.8-62 Units).

Intramuscular injections are administered at up to six sites according to the unit dose. A maximum total dosage administered is 9000 pg (374 Units).

### Example 13

Dosage Regimen for Treating Glabellar, Forehead and Lateral Canthal Lines Using a Modified BoNT/A (SEQ ID NO: 6 Converted into a Di-Chain Form)

Modified BoNT/A is provided as a lyophilised powder in a vial containing 36 ng of modified BoNT/A per vial. The lyophilised powder is reconstituted.

Moderate to severe glabellar, forehead and lateral canthal lines are treated according to the injection regimen shown in FIG. 11.

The unit dose is 20-1500 pg (0.8-62 Units).

Intramuscular injections are administered at up to sixteen sites according to the unit dose. A maximum total dosage administered is 24,000 pg (998 Units).

### Example 14

Safety & Efficacy of Modified BoNT/A (SEQ ID NO: 6 Converted into a Di-Chain Form) in Humans

An integrated phase I/II multicentre, double-blinded, randomised, Dysport and placebo controlled, dose-escalation and dose-finding study to evaluate the safety and efficacy of a modified BoNT/A (SEQ ID NO: 6 converted into a di-chain form) in the treatment of moderate to severe upper facial lines was carried out for adults. Said upper facial lines include glabellar lines, forehead lines and lateral canthal lines. The study included a human sequential dose escalation in cohorts of unique patients with adult upper facial lines. Said upper facial lines include glabellar lines, forehead lines and lateral canthal lines. Each muscle was injected with a unit dose of modified BoNT/A, Dysport, or placebo. 6 cohorts were administered different (increasing) amounts of modified BoNT/A (SEQ ID NO: 6 converted into a di-chain form). The total dose range was 0.1 to 6.0 ng. The modified BoNT/A was injected in up to 16 sites across the upper facial area (five sites in the glabellar lines, five sites in the forehead lines and three sites on each side in lateral canthal lines—see FIG. 11).

Results showed that all unit doses of modified BoNT/A tested and the total dose, (i.e. up to 6,000 pg), were effective, safely tolerated, and no adverse effects were observed, despite the exceptionally high dosage per muscle. This shows that the modified BoNT/A does not diffuse away from the injection site and highlights the exceptional safety profile of modified BoNT/A (SEQ ID NO: 6). Based on these findings, it is considered credible that much higher unit doses can be administered per muscle without resultant adverse effects. Furthermore, given the lack of systemic diffusion of the toxin, it is credible that up to 13-14× the higher unit doses can be administered without safety concerns.

Thus, unit doses of up to 5,000 pg±/−10% each to be administered at up to 16x across the upper facial area (e.g. five sites in the glabellar lines, five sites in the forehead lines and three sites on each side in lateral canthal lines) have been selected for treatment of upper facial lines. The total doses administered during a treatment session will, therefore, be up to 80,000 (+/−10%), respectively.

For example unit doses of up to 5,000 pg±/−10% have been selected for treatment of glabellar lines (two per corrugator, one in the procerus) for a total dose of up to 25,000 pg in the glabellar region which is around 10-fold lower than the NOAEL of 4 ng/kg from both nonclinical safety species (rat and monkey) converting into human dose for 60 kg body weight.

Indeed, as explained in Example 21, 5x injections (two per corrugator, one in the procerus) each of a 4,000 pg unit dose (to provide a total dose of 20,000 pg) was well tolerated.

### Example 15

Dosage Regimen for Treating Glabellar Lines Using a Modified BoNT/A (SEQ ID NO: 6 Converted into a Di-Chain Form)

Modified BoNT/A is provided as a lyophilised powder in a vial containing 36 ng of modified BoNT/A per vial. The lyophilised powder is reconstituted.

Moderate to severe glabellar lines are treated according to the injection regimen shown in FIG. 11. That is, up to two unit doses per corrugator and one in the procerus.

The unit dose is >1754 pg to 5,000 ng. The total dose of modified BoNT/A administered during the treatment is up to 88,000 pg. This is just over 14× greater than the maximum total dosage of Dysport® that can be administered during treatment of facial lines without approaching toxic limits (a concern with conventional treatment regimens). Thus, the clinician is able to tailor treatment to the patient with the knowledge that a total dose 88,000 pg can be administered without any concern of toxicity, thereby allowing the treatment of additional muscles of the subject and/or ensuring each muscle and/or site thereof receives an effective dose.

### Example 16

Dosage Regimen for Treating Glabellar Lines and Forehead Lines Using a Modified BoNT/A (SEQ ID NO: 6 Converted into a Di-Chain Form)

Modified BoNT/A is provided as a lyophilised powder in a vial containing 36 ng of modified BoNT/A per vial. The lyophilised powder is reconstituted.

Moderate to severe glabellar and forehead lines are treated according to the injection regimen shown in FIG. 9. That is, up to two unit doses per corrugator and one in the procerus; and up to five unit doses in the frontalis.

The unit dose is >1754 pg to 5,000 ng. The total dose of modified BoNT/A administered during the treatment is up to 88,000 pg. This is just over 14× greater than the maximum total dosage of Dysport® that can be administered during treatment of facial lines without approaching toxic limits (a concern with conventional treatment regimens). Thus, the clinician is able to tailor treatment to the patient with the knowledge that a total dose 88,000 pg can be administered without any concern of toxicity, thereby allowing the treatment of additional muscles of the subject and/or ensuring each muscle and/or site thereof receives an effective dose.

### Example 17

Dosage Regimen for Treating Lateral Canthal Lines Using a Modified BoNT/A (SEQ ID NO: 6 Converted into a Di-Chain Form)

Modified BoNT/A is provided as a lyophilised powder in a vial containing 36 ng of modified BoNT/A per vial. The lyophilised powder is reconstituted.

Moderate to severe lateral canthal lines are treated according to the injection regimen shown in FIG. 10. That is, up to three unit doses per orbicularis oculi.

The unit dose is >1754 pg to 5,000 ng. The total dose of modified BoNT/A administered during the treatment is up to 88,000 pg. This is just over 14× greater than the maximum total dosage of Dysport® that can be administered during treatment of facial lines without approaching toxic limits (a concern with conventional treatment regimens). Thus, the clinician is able to tailor treatment to the patient with the knowledge that a total dose 88,000 pg can be administered without any concern of toxicity, thereby allowing the treatment of additional muscles of the subject and/or ensuring each muscle and/or site thereof receives an effective dose.

### Example 18

Dosage Regimen for Treating Glabellar, Forehead and Lateral Canthal Lines Using a Modified BoNT/A (SEQ ID NO: 6 Converted into a Di-Chain Form)

Modified BoNT/A is provided as a lyophilised powder in a vial containing 36 ng of modified BoNT/A per vial. The lyophilised powder is reconstituted.

Moderate to severe glabellar, forehead and lateral canthal lines are treated according to the injection regimen shown in FIG. 11. That is, up to two unit doses per corrugator and one in the procerus; and up to five unit doses in the frontalis; up to three unit doses per orbicularis oculi.

The unit dose is >1754 pg to 5,000 ng. The total dose of modified BoNT/A administered during the treatment is up to 88,000 pg. This is just over 14× greater than the maximum total dosage of Dysport® that can be administered during treatment of facial lines without approaching toxic limits (a concern with conventional treatment regimens). Thus, the clinician is able to tailor treatment to the patient with the knowledge that a total dose 88,000 pg can be administered without any concern of toxicity, thereby allowing the treatment of additional muscles of the subject and/or ensuring each muscle and/or site thereof receives an effective dose.

### Example 19

Treatment of a Patient with Upper Facial Lines

John, aged 55, presents with severe glabellar lines, forehead lines and lateral canthal lines. Modified BoNT/A (SEQ ID NO: 6) is administered by way of one or more unit doses (1× unit dose=5000 pg) to the following muscles indicated below:
two unit doses to each corrugator muscle (thus four unit doses across the corrugators) and one site of a procerus muscle for treating glabellar lines,
five unit doses to the frontalis muscle for treating forehead lines; and
three unit doses at the external part of each orbicularis oculi muscle (thus six unit doses across the orbicularis oculi muscles) for treating lateral canthal lines.

The total dose administered is 80,000 ng (+/−10%) modified BoNT/A (SEQ ID NO: 6 converted into a di-chain form).

All lines (wrinkles) are alleviated leaving the skin with a clear and youthful appearance and, owing to the long duration of the modified BoNT/A, Jone does not require further treatment for greater than 9 months. Thus, John receives less frequent injections (e.g. per year) when compared to an equivalent subject administered an unmodified BoNT/A. Additionally, Bill does not exhibit any side-effects owing to the improved safety profile of the modified BoNT/A.

### Example 20

Safety & Efficacy of Modified BoNT/A (SEQ ID NO: 6 Converted into a Di-Chain Form) in Humans

SEQ ID NO: 6 (converted into a di-chain form) was administered to human subjects by way of intramuscular injection. Subjects were administered 2× of a 15,000 pg unit dose (i.e. 30,000 pg total), 2× of a 25,000 pg unit dose (i.e. 50,000 pg total), or 2× of a 36,000 pg unit dose (i.e. 72,000 pg total) of modified BoNT/A (SEQ ID NO: 6 converted into a di-chain form).

Results showed that all unit doses of modified BoNT/A tested were effective at muscle paralysis, safely tolerated, and no adverse effects were observed, despite the exceptionally high dosage per muscle (e.g. for the 25,000 pg and 36,000 pg unit dose). This shows that the modified BoNT/A does not diffuse away from the injection site and highlights the exceptional safety profile of modified BoNT/A (SEQ ID NO: 6 converted into a di-chain form).

The unit doses and the total doses described herein in the context or treating a facial line are well within the dose range shown to be safe and efficacious for this advantageous modified BoNT/A molecules described herein, providing clinicians with flexibility in terms of treatment options that includes utilisation of the advantageous properties and exceptional safety profile of modified BoNT/A (such as SEQ ID NO: 6 converted into a di-chain form).

### Example 21

Safety & Efficacy of Modified BoNT/A (SEQ ID NO: 6 Converted into a Di-Chain Form) in Treatment of Glabellar Lines

SEQ ID NO: 6 (converted into a di-chain form) was administered to human subjects by way of intramuscular injection.

One cohort of subjects were administered 5x injections (2 into each corrugator, 1 into the procerus) of a 2,500 pg unit dose to provide a total dose of 12,500 pg. Another cohort of subjects were administered 5x injections (2 into each corrugator, 1 into the procerus) of a 4,000 pg unit dose to provide a total dose of 20,000 pg.

Glabellar lines were reduced. Results showed that all unit doses of modified BoNT/A tested were safely tolerated, and no adverse effects were observed, despite the exceptionally high dosage per muscle. This shows that the modified BoNT/A does not diffuse away from the injection site and highlights the exceptional safety profile of modified BoNT/A (SEQ ID NO: 6 converted into a di-chain form).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A modified BoNT/A for use in treating facial lines, wherein the modified BoNT/A is administered by intramuscular injection at a plurality of sites of the face of the subject,
wherein the modified BoNT/A is administered by way of a unit dose of greater than 1754 pg (e.g. greater than 1800 pg) of modified BoNT/A per site,
wherein the plurality of sites are selected from:
up to two sites of a corrugator muscle and one site of a procerus muscle for treating glabellar lines,
up to five sites of a frontalis muscle for treating forehead lines, and
up to three sites of an orbicularis oculi muscle for treating lateral canthal lines,
wherein the total dose of modified BoNT/A administered during the treatment is up to 88,000 pg, and
wherein the modified BoNT/A comprises a BoNT/A light-chain and translocation domain (H_{N} domain), and a BoNT/B receptor binding domain (H_{C} domain).

2. The modified BoNT/A for use according to claim 1, wherein the unit dose of modified BoNT/A is greater than 1754 pg and wherein the unit dose of modified BoNT/A is up to 8,000 pg of modified BoNT/A.

3. The modified BoNT/A for use according to claim 1 or claim 2, wherein the unit dose of modified BoNT/A is greater than 1754 pg and wherein the unit dose of modified BoNT/A is up to 7,500 pg of modified BoNT/A.

4. The modified BoNT/A for use according to any one of the preceding claims, wherein the unit dose of modified BoNT/A is greater than 1754 pg and wherein the unit dose of modified BoNT/A is up to 5,000 pg of modified BoNT/A.

5. The modified BoNT/A for use according to any one of the preceding claims, wherein the unit dose is 2000 to 3500 pg of modified BoNT/A, preferably wherein the unit dose is 2250 pg to 3250 pg of modified BoNT/A.

6. The modified BoNT/A for use according to any one of the preceding claims, wherein the unit dose is about 2500 pg of modified BoNT/A, preferably wherein the unit dose is 2500 pg of modified BoNT/A.

7. The modified BoNT/A for use according to any one of claims 1-4, wherein the unit dose is 3500-5500 pg of modified BoNT/A, preferably wherein the unit dose is 3800 pg to 4200 pg of modified BoNT/A.

8. The modified BoNT/A for use according to any one of claim 1-4 or 7, wherein the unit dose is about 4000 pg of modified BoNT/A, preferably wherein the unit dose is 4000 pg of modified BoNT/A.

9. The modified BoNT/A for use according to any one of the preceding claims, wherein the total dose of modified BoNT/A administered during the treatment is up to 80,000 pg.

10. The modified BoNT/A for use according to any one of the preceding claims, wherein the modified BoNT/A has a Safety Ratio of greater than 7, wherein the Safety Ratio is calculated as: dose of toxin required for-10% bodyweight change measured as pg/mouse divided by DAS ED₅₀ measured as pg/mouse, wherein ED₅₀=dose required to produce a DAS score of 2.

11. The modified BoNT/A for use according to any one of the preceding claims, wherein the modified BoNT/A is for use in treating glabellar lines, preferably wherein said modified BoNT/A is administered at two sites of a corrugator muscle and one site of a procerus muscle, more preferably wherein said modified BoNT/A is administered at a total of five sites.

12. The modified BoNT/A for use according to any one of the preceding claims, wherein the modified BoNT/A is for use in treating glabellar lines, preferably wherein said modified BoNT/A is administered at two sites per corrugator muscle and one site of a procerus muscle, more preferably wherein said modified BoNT/A is administered at a total of five sites.

13. The modified BoNT/A for use according to claim 11 or claim 12, wherein a total dose administered for glabellar lines is 12,000 pg to 13,000 pg of modified BoNT/A; preferably wherein the unit dose is 2000 pg to 3000 pg of modified BoNT/A, more preferably wherein the unit dose is 2500 pg of modified BoNT/A.

14. The modified BoNT/A for use according to any one of claims 11-13, wherein a total dose administered for glabellar lines is about 12,500 pg of modified BoNT/A, preferably wherein a total dose administered for glabellar lines is 12,500 pg of modified BoNT/A.

15. The modified BoNT/A for use according to claim 11 or claim 12, wherein a total dose administered for glabellar lines is 19,000 pg to 21,000 pg of modified BoNT/A; preferably wherein the unit dose is 3500 to 4500 pg of modified BoNT/A, more preferably wherein the unit dose is 4000 pg of modified BoNT/A.

16. The modified BoNT/A for use according to any one of claim 11-12 or 15, wherein a total dose administered for glabellar lines is about 20,000 pg of modified BoNT/A, preferably wherein a total dose administered for glabellar lines is 20,000 pg of modified BoNT/A.

17. The modified BoNT/A for use according to any one of claims 1-10, wherein the modified BoNT/A is for treating forehead lines, preferably wherein said modified BoNT/A is administered at five sites of a frontalis muscle.

18. The modified BoNT/A for use according to claim 17, wherein a total dose administered for forehead lines is 12,000 pg to 13,000 pg of modified BoNT/A; preferably wherein the unit dose is 2000 pg to 3000 pg of modified BoNT/A, more preferably wherein the unit dose is 2500 pg of modified BoNT/A.

19. The modified BoNT/A for use according to any one of claims 17-18, wherein a total dose administered for forehead lines is about 12,500 pg of modified BoNT/A, preferably wherein a total dose administered for forehead lines is 12,500 pg of modified BoNT/A.

20. The modified BoNT/A for use according to claim 17, wherein a total dose administered for forehead lines is 19,000 pg to 21,000 pg of modified BoNT/A; preferably wherein the unit dose is 3500 pg to 4500 pg of modified BoNT/A, more preferably wherein the unit dose is 4000 pg of modified BoNT/A.

21. The modified BoNT/A for use according to any one of claim 17 or 20, wherein a total dose administered for forehead lines is about 20,000 pg of modified BoNT/A, preferably wherein a total dose administered for forehead lines is 20,000 pg of modified BoNT/A.

22. The modified BoNT/A for use according to any one of claims 1-10, wherein the modified BoNT/A is for use in treating lateral canthal lines, preferably wherein said modified BoNT/A is administered at three sites of an orbicularis oculi muscle (e.g. at the external part thereof) for treating lateral canthal lines, more preferably wherein said modified BoNT/A is administered at a total of six sites.

23. The modified BoNT/A for use according to any one of claim 1-10 or 22, wherein the modified BoNT/A is for use in treating lateral canthal lines, preferably wherein said modified BoNT/A is administered at three sites per orbicularis oculi muscle (e.g. at the external part of each orbicularis oculi muscle) for treating lateral canthal lines.

24. The modified BoNT/A for use according to claim 22 or claim 23, wherein a total dose administered for lateral canthal lines is 14,000 pg to 16,000 pg of modified BoNT/A; preferably wherein the unit dose is 2000 to 3000 pg of modified BoNT/A, more preferably wherein the unit dose is 2500 pg of modified BoNT/A.

25. The modified BoNT/A for use according to any one of claims 22 to 24, wherein a total dose administered for lateral canthal lines is about 15,000 pg of modified BoNT/A, preferably wherein a total dose administered for lateral canthal lines is 15,000 pg of modified BoNT/A.

26. The modified BoNT/A for use according to any one of claims 22 to 23, wherein a total dose administered for lateral canthal lines is 23,000 pg to 25,000 pg of modified BoNT/A; preferably wherein the unit dose is 3500 pg to 4500 pg of modified BoNT/A, more preferably wherein the unit dose is 4000 pg of modified BoNT/A.

27. The modified BoNT/A for use according to any one of claims 22 to 23, wherein a total dose administered for lateral canthal lines is about 24,000 pg of modified BoNT/A, preferably wherein a total dose administered for lateral canthal lines is 24,000 pg of modified BoNT/A.

28. The modified BoNT/A for use according to any one of claims 1-10, wherein the modified BoNT/A is for use in treating glabellar lines, forehead lines, and lateral canthal lines, preferably wherein:
said modified BoNT/A is administered at two sites of a corrugator muscle and one site of a procerus muscle for treating glabellar lines;
said modified BoNT/A is administered at five sites of a frontalis muscle; and
said modified BoNT/A is administered at three sites at the external part of an orbicularis oculi muscle for treating lateral canthal lines.

29. The modified BoNT/A for use according to any one of the preceding claims, wherein the modified BoNT/A is for use in treating glabellar lines, forehead lines, and lateral canthal lines, preferably wherein:
said modified BoNT/A is administered at two sites per corrugator muscle and one site of a procerus muscle for treating glabellar lines;
said modified BoNT/A is administered at five sites of a frontalis muscle; and
said modified BoNT/A is administered at three sites per orbicularis oculi muscle (preferably at the external part of each orbicularis oculi muscle), for treating lateral canthal lines.

30. The modified BoNT/A for use according to claim 28 or claim 29, wherein a total dose for treating glabellar lines, forehead lines, and lateral canthal lines is 43,000 to 44,000 pg of modified BoNT/A; preferably wherein the unit dose is 2000 pg to 3000 pg of modified BoNT/A, more preferably wherein the unit dose is 2500 pg of modified BoNT/A.

31. The modified BoNT/A for use according to any one of claims 28-30, wherein a total dose for treating glabellar lines, forehead lines, and lateral canthal lines is about 44,000 of modified BoNT/A, preferably wherein a total dose for treating glabellar lines, forehead lines, and lateral canthal lines is 44,000 pg of modified BoNT/A.

32. The modified BoNT/A for use according to claim 28 or claim 29, wherein a total dose for treating glabellar lines, forehead lines, and lateral canthal lines is 63,000 to 65,000 pg of modified BoNT/A; preferably wherein the unit dose is 3500 pg to 4500 pg of modified BoNT/A, more preferably wherein the unit dose is 4000 pg of modified BoNT/A.

33. The modified BoNT/A for use according to claim 28, 29, or 32, wherein a total dose for treating glabellar lines, forehead lines, and lateral canthal lines is about 64,000 pg of modified BoNT/A, preferably wherein a total dose for treating glabellar lines, forehead lines, and lateral canthal lines is 64,000 pg of modified BoNT/A.

34. The modified BoNT/A for use according to any one of the preceding claims, wherein said modified BoNT/A is administered to 8, 10, 12, 14 or 16 sites.

35. The modified BoNT/A for use according to any one of the preceding claims, wherein said modified BoNT/A is administered to 7, 9, 11, 13 or 15 sites.

36. The modified BoNT/A for use according to any one of the preceding claims, wherein the modified BoNT/A comprises a polypeptide sequence having at least 70%, 75%, 80%, 85%, 90% or 95% sequence identity to SEQ ID NO: 6; preferably wherein the modified BoNT/A comprises a polypeptide sequence having at least 70% sequence identity to SEQ ID NO: 6.

37. The modified BoNT/A for use according to any one of the preceding claims, wherein the modified BoNT/A comprises a BoNT/A light-chain and translocation domain, and a BoNT/B H_{C} domain, and wherein 24.04 pg of the modified BoNT/A corresponds to the calculated median lethal dose (LD₅₀) in mice (e.g. and wherein 1 Unit is an amount of the modified BoNT/A that corresponds to the calculated median lethal dose (LD₅₀) in mice).

38. A unit dosage form of modified botulinum neurotoxin A (BoNT/A), the unit dosage form comprising:
a. greater than 73 Units of modified BoNT/A, wherein 1 Unit is an amount of the modified BoNT/A that corresponds to the calculated median lethal dose (LD₅₀) in mice; or
b. greater than 1754 pg (preferably greater than 1800 pg) of modified BoNT/A; and
c. optionally a pharmaceutically acceptable carrier, excipient, adjuvant, and/or salt,
wherein the modified BoNT/A comprises a BoNT/A light-chain and translocation domain (H_{N} domain), and a BoNT/B receptor binding domain (H_{C} domain).

39. The unit dosage form according to claim 38, comprising:
a. greater than 73 Units and up to 332.8 Units of modified BoNT/A; or
b. greater than 1754 pg (preferably greater than 1800 pg) and up to 8000 pg of modified BoNT/A.

40. The unit dosage for according to claim 38 or claim 39, wherein the unit dosage form comprises 2000 pg to 3500 pg of modified BoNT/A, preferably 2250 pg to 3250 pg of modified BoNT/A.

41. The unit dosage form according to any one of claims 38 to 39, wherein the unit dosage form comprises about 2500 pg of modified BoNT/A, preferably wherein the unit dosage form comprises 2500 pg of modified BoNT/A.

42. The unit dosage form according to claim 38 or claim 39, wherein the unit dosage form comprises 3500 pg to 5500 pg of modified BoNT/A, preferably 3800 pg to 4200 pg of modified BoNT/A.

43. The unit dosage form according to any one of claim 38, 39 or 42, wherein the unit dosage form comprises about 4000 pg of modified BoNT/A, preferably wherein the unit dosage form comprises 4000 pg of modified BoNT/A.

44. A kit comprising:
a. the unit dosage form according to any one of claims 38-43; and
b. instructions for use of the same in treating facial lines (e.g. glabellar lines, forehead lines and/or lateral canthal lines); and
c. optionally a diluent.


SEQUENCE LISTING (13 sequences)

SEQ ID NO: 1 - DNA, 3888 bp, Clostridium botulinum
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - protein, 1315 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 4 - protein, 1327 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 5 - protein, 1314 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 6 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 7 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 8 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 9 - protein, 10 aa, synthetic construct
TKSLDKGYNK

SEQ ID NO: 10 - protein, 8 aa, synthetic construct
SLDKGYNK

SEQ ID NO: 11 - protein, 437 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 12 - protein, 439 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTK

SEQ ID NO: 13 - protein, 856 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE